(19) United States
(12) Patent Application Publication
(10) Pub. No.: US 2010/0279995 A1
(43) Pub. Date: Nov. 4, 2010

(54) Novel Ansamycin Derivatives

(76) Inventors: Alexander R. Guiblin, Essex (GB); Barrie Wilkinson, Essex (GB); Steven J. Moss, Essex (GB); Ming-Qiang Zhang, Essex (GB); Alison D. McElhinney, Essex (GB); Christine J. Martin, Essex (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/063,791

(22) PCT Filed: Sep. 1, 2006
(86) PCT No.: PCT/EP06/65941

§ 371 Date: Jul. 21, 2010

(30) Foreign Application Priority Data
Sep. 2, 2005 (GB) 0517886.8

(51) Int. Cl. A61K 31/395; C07D 225/04; A61P 35/00

(52) U.S. Cl. 514/183; 540/461

(57) ABSTRACT

There are provided inter alia derivatives of a benzenoid ansamycin which contain a 1,4-dihydroxyphenyl moiety bearing at position 6 an amino carboxy substituent, in which position 2 and the carboxy substituent at position 6 are connected by an aliphatic chain of varying length characterised in that one or both of the 1-hydroxy and the 4-hydroxy position(s) of the phenyl ring are independently derivatised by an aminoalkyleneaminocarbonyl group, which alkylene group, which may optionally be substituted by alkyl groups, has a chain length of 2 or 3 carbons and which derivatising group(s) increase the water solubility and/or the bioavailability of the parent molecule but which are capable of being removed in-vivo. Such compounds are described for the treatment of cancer or B-cell malignancies.

### INTRODUCTION

The present invention relates to derivatives of ansamycin compounds that are useful, e.g. in the treatment of cancer or B-cell malignancies, in particular the derivatives are pro-drugs of ansamycin compounds. The present invention also provides methods for the production of these compounds and their use in medicine, in particular in the treatment and/or prophylaxis of cancer or B-cell malignancies.

### BACKGROUND OF THE INVENTION

The development of highly specific anticancer drugs with low toxicity and favourable pharmacokinetic characteristics comprises a major challenge in anticancer therapy.

The 90 kDa heat shock protein (Hsp90) is an abundant molecular chaperone involved in the folding and assembly of proteins, many of which are involved in signal transduction pathways (for reviews see Neckers, 2002; Sreedhar et al., 2004a; Wegele et al., 2004 and references therein). So far nearly 50 of these so-called client proteins have been identified and include steroid receptors, non-receptor tyrosine kinases e.g. src family, cyclin-dependent kinases e.g. cdk4 and cdk6, the cystic transmembrane regulator, nitric oxide synthase and others (Donzé and Picard, 1999; McLaughlin et al., 2002; Chiosis et al., 2004; Wegele et al., 2004; http://www.picard.ch/downloads/Hsp90interactors.pdf). Furthermore, Hsp90 plays a key role in stress response and protection of the cell against the effects of mutation (Bagatell and Whitesell, 2004; Chiosis et al., 2004). The function of Hsp90 is complicated and it involves the formation of dynamic multi-enzyme complexes (Bohen, 1998; Liu et al., 1999; Young et al., 2001; Takahashi et al., 2003; Sreedhar et al., 2004; Wegele et al., 2004). Hsp90 is a target for inhibitors (Fang et al., 1998; Liu et al., 1999; Blagosklonny, 2002; Neckers, 2003; Takahashi et al., 2003; Beliakoff and Whitesell, 2004; Wegele et al., 2004) resulting in degradation of client proteins, cell cycle dysregulation and apoptosis. More recently, Hsp90 has been identified as an important extracellular mediator for tumour invasion (Eustace et al., 2004). Hsp90 was identified as a new major therapeutic target for cancer therapy which is mirrored in the intense and detailed research about Hsp90 function (Blagosklonny et al., 1996; Neckers, 2002; Workman and Kaye, 2002; Beliakoff and Whitesell, 2004; Harris et al., 2004; Jez et al., 2003; Lee et al., 2004) and the development of high-throughput screening assays (Carreras et al., 2003; Rowlands et al., 2004). Hsp90 inhibitors include compound classes such as ansamycins, macrolides, purines, pyrazoles, coumarin antibiotics and others (for review see Bagatell and Whitesell, 2004; Chiosis et al., 2004 and references therein).

The benzenoid ansamycins are a broad class of chemical structures characterised by an aliphatic ring of varying length joined either side of an aromatic ring structure. Naturally occurring ansamycins include: macbecin and 18,21-dihydromacbecin (also known as macbecin I and macbecin II respectively) (1 & 2; Tanida et al., 1980), geldanamycin (3; DeBoer et al., 1970; DeBoer and Dietz, 1976; WO 03/106653 and references therein), and the herbimycin family (4; 5, 6, Omura et al., 1979, Iwai et al., 1980 and Shibata et al, 1986a, WO 03/106653 and references therein).

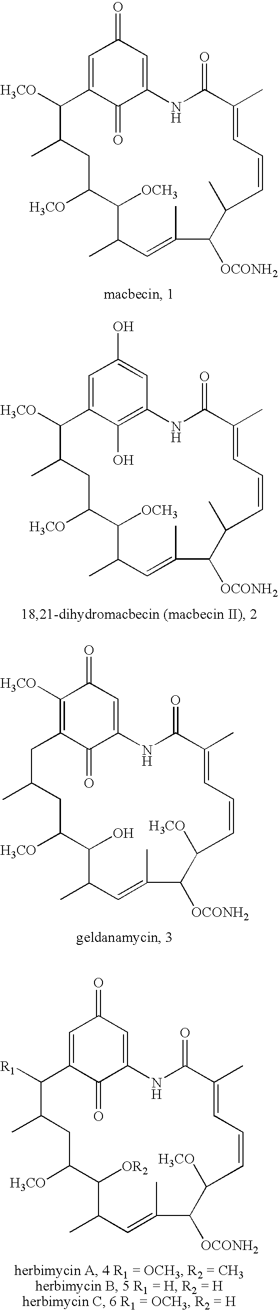

Ansamycins were originally identified for their antibacterial and antiviral activity, however, recently their potential utility as anticancer agents has become of greater interest (Beliakoff and Whitesell, 2004). Many Hsp90 inhibitors are currently being assessed in clinical trials (Csermely and Soti, 2003; Workman, 2003). In particular, geldanamycin has nanomolar potency and apparent specificity for aberrant protein kinase dependent tumour cells (Chiosis et al., 2003; Workman, 2003).

It has been shown that treatment with Hsp90 inhibitors enhances the induction of tumour cell death by radiation and increased cell killing abilities (e.g. breast cancer, chronic myeloid leukemia and non-small cell lung cancer) by combination of Hsp90 inhibitors with cytotoxic agents has also been demonstrated (Neckers, 2002; Beliakoff and Whitesell, 2004). The potential for anti-angiogenic activity is also of interest: the Hsp90 client protein HIF-1α plays a key role in the progression of solid tumours (Hur et al., 2002; Workman and Kaye, 2002; Kaur et al., 2004).

Hsp90 inhibitors also function as immunosuppressants and are involved in the complement-induced lysis of several types of tumour cells after Hsp90 inhibition (Sreedhar et al., 2004). Treatment with Hsp90 inhibitors can also result in induced superoxide production (Sreedhar et al., 2004a) associated with immune cell-mediated lysis (Sreedhar et al., 2004). The use of Hsp90 inhibitors as potential anti-malaria drugs has also been discussed (Kumar et al., 2003). Furthermore, it has been shown that geldanamycin interferes with the formation of complex glycosylated mammalian prion protein PrP^{c} (Winklhofer et al., 2003).

As described above, ansamycins are of interest as potential anticancer and anti-B-cell malignancy compounds, however the currently available ansamycins exhibit poor pharmacological or pharmaceutical properties, for example they show poor water solubility, poor metabolic stability, poor bioavailability or poor formulation ability (Goetz et al., 2003; Workman 2003; Chiosis 2004). Both herbimycin A and geldanamycin were identified as poor candidates for clinical trials due to their strong hepatotoxicity (review Workman, 2003) and geldanamycin was withdrawn from Phase I clinical trials due to hepatotoxicity (Supko et al., 1995, WO 03/106653)

Geldanamycin was isolated from culture filtrates of Streptomyces hygroscopicus and shows strong activity in vitro against protozoa and weak activity against bacteria and fungi. In 1994 the association of geldanamycin with Hsp90 was shown (Whitesell et al., 1994). The biosynthetic gene cluster for geldanamycin was cloned and sequenced (Allen and Ritchie, 1994; Rascher et al., 2003; WO 03/106653). The DNA sequence is available under the NCBI accession number AY179507. The isolation of genetically engineered geldanamycin producer strains derived from S. hygroscopicus subsp. duamyceticus JCM4427 and the isolation of 4,5-dihydro-7-O-descarbamoyl-7-hydroxygeldanamycin and 4,5-dihydro-7-O-descarbamoyl-7-hydroxy-17-O-demethylgeldanamycin were described recently (Hong et al., 2004). By feeding geldanamycin to the herbimycin producing strain Streptomyces hygroscopicus AM-3672 the compounds 15-hydroxygeldanamycin, the tricyclic geldanamycin analogue KOSN-1633 and methyl-geldanamycinate were isolated (Hu et al., 2004). The two compounds 17-formyl-17-demethoxy-18-O-21-O-dihydrogeldanamycin and 17-hydroxymethyl-17-demethoxygeldanamycin were isolated from S. hygroscopicus NRRL 3602 containing plasmid pKOS279-78 with various genes from the herbimycin producing strain Streptomyces hygroscopicus AM-3672 (Hu et al., 2004).

In 1979 the ansamycin antibiotic herbimycin A was isolated from the fermentation broth of Streptomyces hygroscopicus strain No. AM-3672 and named according to its potent herbicidal activity. The antitumour activity was established by using cells of a rat kidney line infected with a temperature sensitive mutant of Rous sarcoma virus (RSV) for screening for drugs that reverted the transformed morphology of the these cells (for review see Uehara, 2003). Herbimycin A was postulated as acting primarily through the binding to Hsp90 chaperone proteins but the direct binding to the conserved cysteine residues and subsequent inactivation of kinases was also discussed (Uehara, 2003).

Chemical derivatives have been isolated and compounds with altered substituents at C19 of the benzoquinone nucleus and halogenated compounds in the ansa chain showed less toxicity and higher antitumour activities than herbimycin A (Omura et al., 1984; Shibata et al., 1986b). The sequence of the herbimycin biosynthetic gene cluster was identified in WO 03/106653 and in a recent paper (Rascher et al, 2005).

The ansamycin antibiotics macbecin (1) and 18,21-dihydromacbecin (2) (C-14919E-1 and C-14919E-1), identified by their antifungal and antiprotozoal activity, were isolated from the culture supernatants of Nocardia sp No. C-14919 (Actinosynnema pretiosum subsp pretiosum ATCC 31280) (Tanida et al., 1980; Muroi et al., 1980; U.S. Pat. No. 4,315,989 and U.S. Pat. No. 4,187,292). 18,21-Dihydromacbecin is characterized by containing the hydroquinone form of the nucleus. Both macbecin and 18,21-dihydromacbecin were shown to possess similar antibacterial and antitumour activities against cancer cell lines such as the murine leukemia P388 cell line (Ono et al., 1982). Reverse transcriptase and terminal deoxynucleotidyl transferase activities were not inhibited by macbecin (Ono et al., 1982). The Hsp90 inhibitory function of macbecin has been reported in the literature (Bohen, 1998; Liu et al., 1999). The conversion of macbecin and 18,21-dihydromacbecin after adding to a microbial culture broth into a compound with a hydroxy group instead of a methoxy group at a certain position or positions is described in patents U.S. Pat. No. 4,421,687 and U.S. Pat. No. 4,512,975.

During a screen of a large variety of soil microorganisms, the antibiotics TAN-420A to E were identified from producer strains belonging to the genus Streptomyces (7-11, EP 0 110 710).

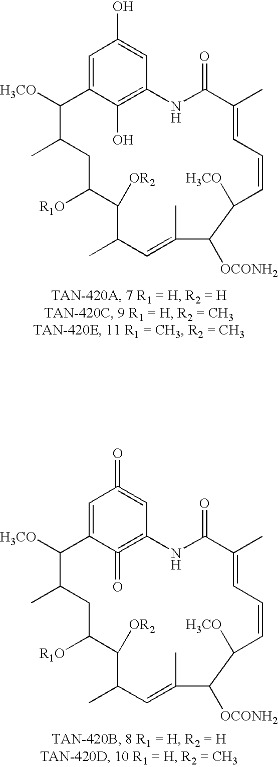

In 2000, the isolation of the geldanamycin related, non-benzoquinone ansamycin metabolite reblastatin from cell cultures of Streptomyces sp. S6699 and its potential therapeutic value in the treatment of rheumatoid arthritis was described (Stead et al., 2000).

A further Hsp90 inhibitor, distinct from the chemically unrelated benzoquinone ansamycins is Radicicol (monorden) which was originally discovered for its antifungal activity from the fungus Monosporium bonorden (for review see Uehara, 2003) and the structure was found to be identical to the 14-membered macrolide isolated from Nectria radicicola. In addition to its antifungal, antibacterial, anti-protozoan and cytotoxic activity it was subsequently identified as an inhibitor of Hsp90 chaperone proteins (for review see Uehara, 2003; Schulte et al., 1999). The anti-angiogenic activity of radicicol (Hur et al., 2002) and semi-synthetic derivates thereof (Kurebayashi et al., 2001) has also been described.

Recent interest has focused on 17-amino derivatives of geldanamycin as a new generation of ansamycin anticancer compounds (Bagatell and Whitesell, 2004), for example 17-(allylamino)-17-desmethoxy geldanamycin (17-AAG, 12) (Hostein et al., 2001; Neckers, 2002; Nimmanapalli et al., 2003; Vasilevskaya et al., 2003; Smith-Jones et al., 2004) and 17-desmethoxy-17-N,N-dimethylaminoethylamino-geldanamycin (17-DMAG, 13) (Egorin et al., 2002; Jez et al., 2003). More recently geldanamycin was derivatised on the 17-position to create 17-geldanmycin amides, carbamates, ureas and 17-arylgeldanamycin (Le Brazidec et al., 2003). A library of over sixty 17-alkylamino-17-demethoxygeldanamycin analogues has been reported and tested for their affinity for Hsp90 and water solubility (Tian et al., 2004). A further approach to reduce the toxicity of geldanamycin is the selective targeting and delivering of an active geldanamycin compound into malignant cells by conjugation to a tumour-targeting monoclonal antibody (Mandler et al., 2000).

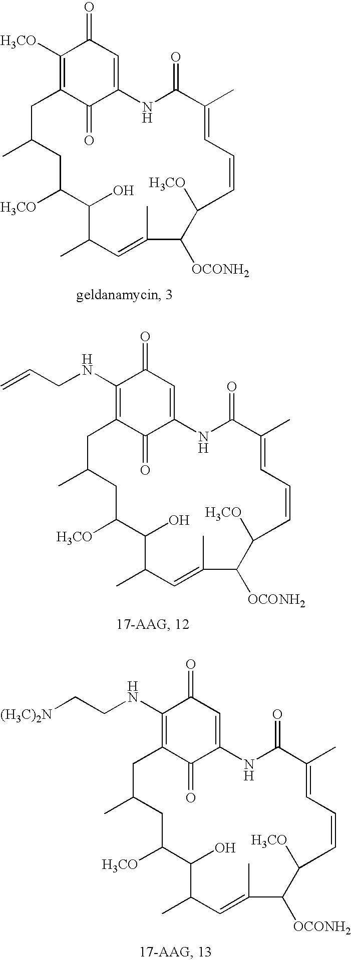

Whilst these derivatives exhibit reduced hepatotoxicity they still have only limited water solubility and require the use of a solubilising carrier (e.g. Cremophore®, DMSO-egg lecithin), which itself may result in side-effects in some patients.

Therefore, there remains a need to identify novel ansamycins with improved water solubility which will have an improved pharmacological profile and reduced side-effect profile for administration. The present invention discloses novel ansamycin derivatives which are pro-drugs and may be cleaved, chemically or enzymatically, to the parent molecule and which generally have improved pharmaceutical properties compared with the presently available ansamycins; in particular they show improvements in respect of one or more of the following properties: water solubility, bioavailability and formulation ability.

### SUMMARY OF THE INVENTION

The present invention provides derivatives of ansamycins, methods for the preparation of these compounds, intermediates thereto and methods for the use of these compounds in medicine. In particular the derivatives of ansamycins are pro-drugs.

In its broadest aspect the present invention provides derivatives of benzenoid ansamycins containing self-cleaving and water solubilizing derivatising group(s) at positions 18 and/or 21 of the parent molecule. These groups are designed to be chemically cleaved to produce the bioactive parent molecule, alternatively cleavage may occur via enzymatic means.

Thus the invention relates to derivatives of a benzenoid ansamycin which contain a 1,4-dihydroxyphenyl moiety bearing at position 6 an amino carboxy substituent, in which position 2 and the carboxy substituent at position 6 are connected by an aliphatic chain of varying length characterised in that one or both of the 1-hydroxy and the 4-hydroxy position(s) of the phenyl ring are independently derivatised by an aminoalkyleneaminocarbonyl group, which alkylene group (which may optionally be substituted by alkyl eg methyl groups) has a chain length of 2 or 3 carbons and which derivatising group(s) increase the water solubility and/or the bioavailability of the parent molecule but which are capable of being removed in-vivo eg by self-cleavage.

In this context the “parent molecule” means the corresponding molecule bearing an underivatised hydroxyl group at positions 1 and 4 of the phenyl ring (i.e. hydroquinone) or the benzoquinone form thereof.

In a more specific aspect the present invention provides derivatives of ansamycins according to the formulas (IA-IC) below, or a pharmaceutically acceptable salt thereof:

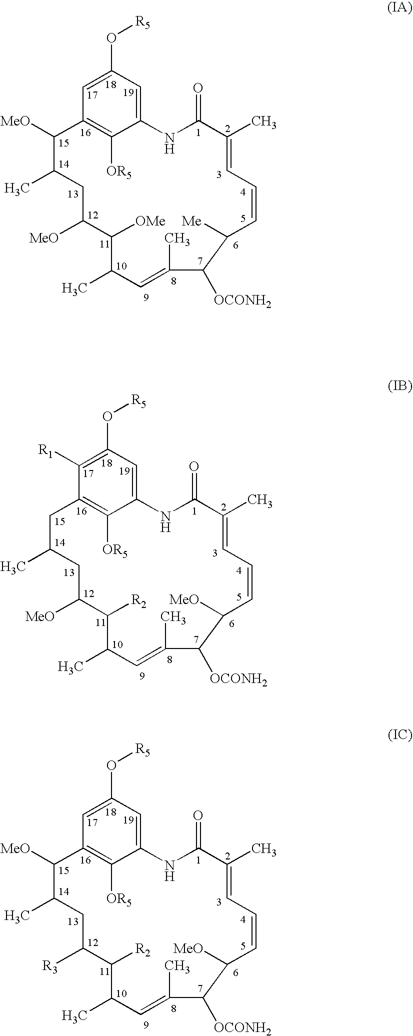

wherein:
R₁ represents H, OH, OMe, —NHCH₂CH═CH₂ or —NHCH₂CH₂N(CH₃)₂;
R₂ represents OH, or keto;
R₃ represents OH or OMe;
R₅ represents H or

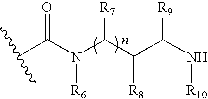

wherein:
n represents 0 or 1;
R₆ represents H, Me, Et or iso-propyl;
R₇, R₈ and R₉ each independently represent H or a C1-C4 branched or linear chain alkyl group;
or R₇ and R₈, or R₈ and R₉, may be connected so as to form a 6-membered carbocyclic ring;
R₁₀ represents H or a C1-C4 branched or linear chain alkyl group;
provided however that the R₅ moieties are not both H and that when neither R₅ moiety represents H then the two R₅ moieties are the same.

The above structure shows a representative tautomer and the invention embraces all tautomers of the compounds of formula (IA), (IB) and (IC) for example keto compounds where enol compounds are illustrated and vice versa.

Compounds of formula (IA), (IB) and (IC) are referred to collectively in the foregoing as compounds of formula (I).

In a further aspect, the present invention provides ansamycin derivatives such as compounds of formula (I) or a pharmaceutically acceptable salt thereof, for use as a pharmaceutical.

### Definitions

The articles “a” and “an” are used herein to refer to one or to more than one (i.e. at least one) of the grammatical objects of the article. By way of example “an analogue” means one analogue or more than one analogue.

As used herein the term “analogue(s)” refers to chemical compounds that are structurally similar to another but which differ slightly in composition (as in the replacement of one atom by another or in the presence or absence of a particular functional group).

As used herein, the term “cancer” refers to a malignant new growth that arises from epithelium, found in skin or, more commonly, the lining of body organs, for example, breast, prostate, lung, kidney, pancreas, stomach or bowel. A cancer tends to infiltrate into adjacent tissue and spread (metastasise) to distant organs, for example to bone, liver, lung or the brain. As used herein the term cancer includes both metastatic tumour cell types, such as but not limited to, melanoma, lymphoma, leukemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, gliobastoma, primary liver cancer and ovarian cancer.

As used herein, the term “bioavailability” refers to the degree to which or rate at which a drug or other substance is absorbed or becomes available at the site of biological activity after administration. This property is dependent upon a number of factors including the solubility of the compound, rate of absorption in the gut, the extent of protein binding and metabolism etc. Various tests for bioavailability that would be familiar to a person of skill in the art are described herein (see also Egorin et al. (2002).

As used herein the term “B-cell malignancies” includes a group of disorders that include chronic lymphocytic leukemia (CLL), multiple myeloma, and non-Hodgkin's lymphoma (NHL). They are neoplastic diseases of the blood and blood forming organs. They cause bone marrow and immune system dysfunction, which renders the host highly susceptible to infection and bleeding.

The term “pro-drug” as used in this application refers to a precursor or derivative form of a pharmaceutically active substance that has an improved formulation profile compared to the parent drug, e.g. it may be less cytotoxic or more soluble compared to the parent drug, and it is capable of being activated (e.g. cleaved chemically or enzymatically) or otherwise converted into the more active parent form (see, for example, Wilman D. E. V. (1986) “Pro-drugs in Cancer Chemotherapy” Biochemical Society Transactions 14, 375-382 (615th Meeting, Belfast) and Stella V. J. et al (1985) “Pro-drugs: A Chemical Approach to Targeted Drug Delivery” Directed Drug Delivery R. Borchardt et al (ed.) pages 247-267 (Humana Press).

The term “water solubility” as used in this application refers to solubility in aqueous media, e.g. phosphate buffered saline (PBS) at pH 7.4, or in 5% glucose solution. Tests for water solubility are given below in the Examples as “water solubility assay”.

As used herein, the term “ansamycin derivative” refers to a benzenoid ansamycin derivative referred to above as representing the invention in its broadest aspect, for example a compound according to formula (I) above, or a pharmaceutically acceptable salt thereof. These compounds are also referred to as “compounds of the invention” or “derivatives of ansamycins” and these terms are used interchangeably in the present application.

The pharmaceutically acceptable salts of compounds of the invention such as the compounds of formula (I) include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Hydrochloric acid salts are of particular interest. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts. References hereinafter to a compound according to the invention include both compounds of formula (I) and their pharmaceutically acceptable salts.

Alkyl, alkenyl and alkynyl groups may be straight chain or branched.

Examples of alkyl eg C1-C4 alkyl groups include methyl, ethyl, n-propyl, i-propyl and n-butyl.

As used herein the terms “18,21-dihydromacbecin” and “macbecin II” (the hydroquinone of macbecin I) are used interchangeably.

### FIGURE LEGEND

FIG. 1 Graph showing the decay of the parent compound and the relative amounts of released compounds in a cleavage assay.

FIG. 2 Graph showing group median relative tumour volumes over time in PRXF DU-145 xenografts.

### DESCRIPTION OF THE INVENTION

Strategies to improve physical properties of drug candidates e.g. bioavailability typically employ a pro-drug precursor which relies upon environmental influence such as enzymatic hydrolysis to release active parent drug. However, due to variation between individuals, release of active drug at a required rate in vivo may not be achievable.

The present invention provides derivatives of ansamycins with an alternative method of rate controlled active drug release. This approach utilises self-cleaving of an incorporated amino side chain triggered at physiological pH via an intramolecular cyclisation-elimination reaction. Such intramolecular attack by a terminal amino group upon a carbamate functionality generates a cyclic urea fragment and leads to parent drug release.

The rate of drug release is governed by chemical cyclisation rate constants and associated substituents rather than by external influence.

Whilst it is intended that the compounds of the invention are capable of chemically mediated self-cleavage, it is also possible that they are substrates for enzymatic cleavage and this is also encompassed within the scope of the present invention.

Thus, the present invention provides derivatives of ansamycins, as set out above, methods for the preparation of these compounds, intermediates thereto and methods for the use of these compounds in medicine.

In one example set of compounds of formula IA-IC, R₆ represents H, Me or Et. In a further example set of compounds of formula IA-IC, R₁₀ represents a C1-4 branched or linear alkyl group. In a further example set of compounds of formula IA-IC, R₆ represents H, Me or Et and R₁₀ represents a C1-4 branched or linear alkyl group.

Each of the R₅ groups will be the same or one will be H whilst the other is substituted as described herein. In one example aspect one of the R₅ groups is H; in an alternative example aspect neither R₅ group is H. Preferably the C21 R₅ group is H.

Preferably R₆ represents Me. Alternatively, preferably R₆ represents Et.

Preferably R₁₀ represents Me. Alternatively, preferably R₁₀ represents Et.

Preferably R₇ represents H. Preferably R₈ represents H. Preferably R₉ represents H.

When R₇ and R₈ or R₈ and R₉ are connected to form a six-membered carbocyclic ring that ring may suitably be a cyclohexyl ring.

Preferably n=0.

The stereochemistry of side chains relative to the ansamycin ring preferably follows that of the corresponding parent compounds (i.e. macbecin, geldanamycin, herbimycin A, see eg structure shown above Table 4).

The compound of formula (I) may, for example, represent a derivative of the following compounds:
macbecin (formula (IA));
geldanamycin (formula (IB) in which R₁ represents OMe, particularly when R₂ represents OH);
herbimycin B (formula (IB) in which R₁ represents H, particularly when R₂ represents OH);
17-AAG (formula (IB) in which R₁ represents NHCH₂CH═CH₂, particularly when R₂ represents OH);
17-DMAG (formula (IB) in which R₁ represents NHCH₂CH₂NMe₂, particularly when R₂ represents OH);
herbimycin A (formula (IC) in which R₂ represents OMe and R₃ represents OMe); or
herbimycin C (formula (IC) in which R₂ represents OH and R₃ represents OMe)

In general, the compounds of the invention are prepared by semi-synthetic derivatisation of the ansamycin family of compounds.

Thus a process for preparing a compound of formula (I) or a pharmaceutically acceptable salt thereof comprises:

(a) preparing a compound of formula (I) in which neither R₅ moiety is H by reacting a compound of formula (IIA), (IIB) or (IIC):

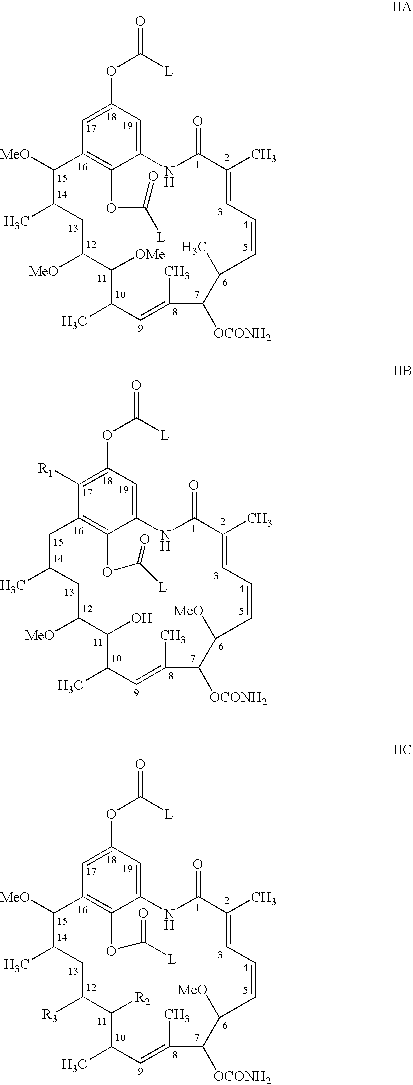

wherein L is a leaving group

or a protected derivative thereof, with a compound of formula (H)

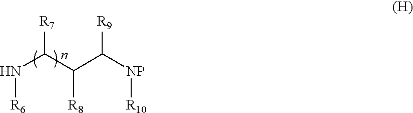

wherein P represents a protecting group; or

(b) preparing a compound of formula (I) in which the C21 R₅ moiety is H by reacting a compound of formula (IID), (IIE) or (IIF):

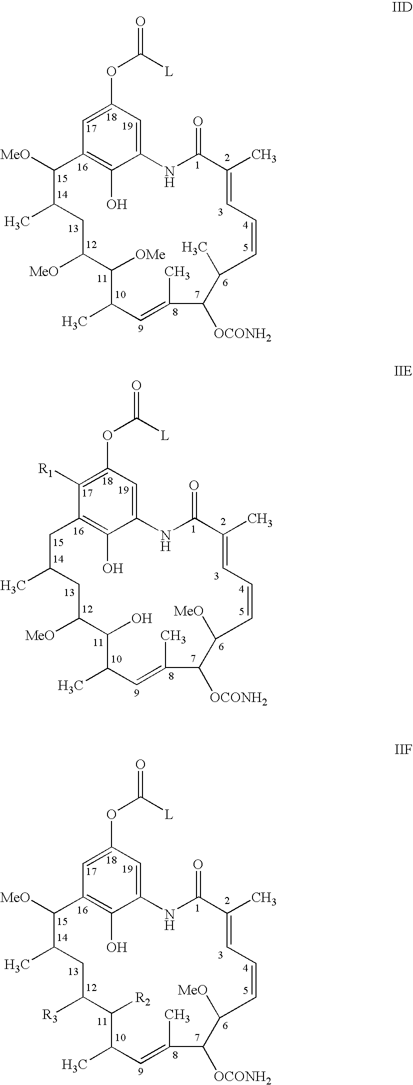

wherein L is a leaving group

or a protected derivative thereof, with a compound of formula (H)

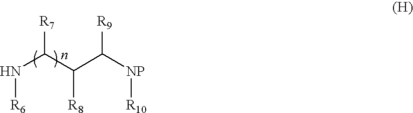

wherein P represents a protecting group; or

(c) converting a compound of formula (I) or a salt thereof to another compound of formula (I) or another pharmaceutically acceptable salt thereof; or

(d) deprotecting a protected compound of formula (I).

In the foregoing text the compounds of formula (IIA), (IIB), (IIC), (IID), (IIE) and (IIF) are referred to collectively as compounds of formula (II).

In processes (a) and (b), exemplary leaving groups L include halogen (eg chlorine, bromine), alkoxy (eg C1-4alkoxy), aryl (eg phenoxy or substituted phenoxy such as 4-nitrophenoxy) or alkylaryl (eg C1-4alkylaryl eg benzyloxy). Preferably L represents 4-nitrophenoxy. Exemplary protecting groups P include t-butyloxycarbonyl (“Boc”) and the trityl group.

The reaction of compounds of formula (II) with compound of formula (H) may be performed under conventional conditions known per se for carbamate formation eg reflux of the ingredients in an inert solvent such as dichloromethane with water work-up.

Compounds of formula (II), or a protected derivative thereof, may be prepared by reacting a compound of formula IIIA-IIIC:

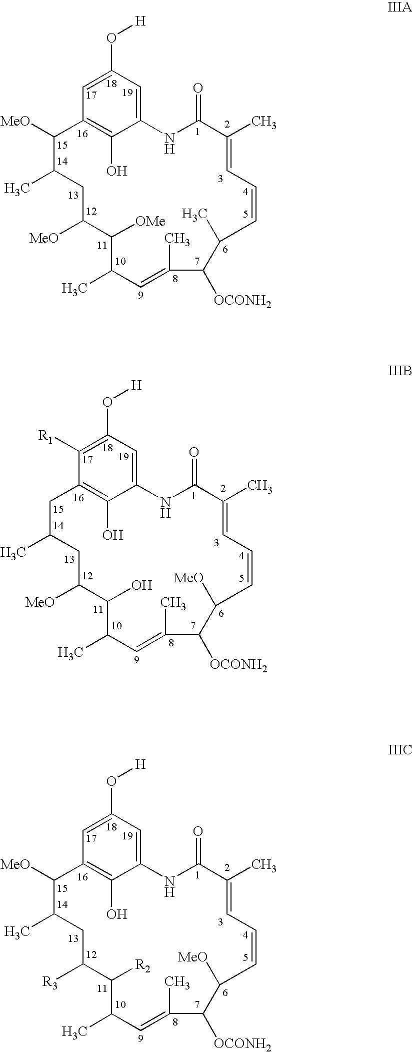

or a protected derivative thereof, with a compound of formula (J):

L′-CO-L (J)

wherein L′ represents a leaving group, preferably one which is more labile than L. Exemplary L′ groups are as described for L, above. A preferred compound of formula J is 4-nitrophenylchloroformate.

The reaction of compounds of formula (III) with compound of formula (J) may be performed under conventional conditions known per se eg reflux of the ingredients in an inert solvent such as dichloromethane.

Since the C18 OH group is more reactive than the C21 OH group, compounds of formula (IID)-(IIF) may be obtained by reacting the corresponding compound of formula (IIIA)-(IIIC) with a slight excess of the compound of formula J. Compounds of formula (IIA)-(IIC) may be obtained by reacting the corresponding compound of formula (IIIA)-(IIIC) with a greater than two times excess of the compound of formula J.

Compounds of formula IIIA-IIIC (hereinafter “compounds of formula (III)”) and protected derivatives thereof may be prepared as follows:

Firstly, the naturally occurring ansamycins for use as templates may be obtained via direct fermentation of strains which produce the desired compound. A person of skill in the art will be able to culture a producer strain under suitable conditions for the production and isolation of the natural product template. The strains listed in Table 1 are examples of producer strains for the natural product templates, but a person of skill in the art will appreciate that there may be alternative strains available that will produce the same compound under appropriate conditions.

TABLE 1
producer strains

| Natural Product | |
|---|---|
| Template | Producer strain(s) |
| Macbecin and 18,21- | Actinosynnema pretiosum subsp pretiosum |
| dihydromacbecin | ATCC31280 |
| | Actinosynnema mirum DSM43827 |
| Geldanamycin | Streptomyces. hygroscopicus var geldanus |
| | NRRL 3602 |
| | Streptomyces violaceusniger DSM40699 |
| | Streptomyces sp. DSM4137 |
| Herbimycin A-C | Streptomyces. hygroscopicus AM-3672 |
| TAN 420A-E | Streptomyces sp. No C-41125, C-41206 |

Alternatively, the compounds may be commercially available; Table 2 lists the compounds that may be purchased together with their catalogue numbers:

TABLE 2
commercially available compounds of formula (I)

| Natural Product Template | Supplier (Catalogue #) |
|---|---|
| Geldanamycin | A.G. Scientific, Inc. (G-1047) |
| Herbimycin A | A.G. Scientific, Inc. (H1050) |
| 17-AAG | A.G. Scientific, Inc. (A1256) |
| 17-DMAG | Invivogen (17DMAG) |

In addition to the specific methods and references provided herein a person of skill in the art may also consult standard textbook references for synthetic methods, including, but not limited to Vogel's Textbook of Practical Organic Chemistry (Furniss et al., 1989) and March's Advanced Organic Chemistry (Smith and March, 2001).

The naturally occurring ansamycins exist and can be isolated predominantly in their benzoquinone form. In some cases the hydroquinone form may be isolated from the fermentation broth. If the benzoquinone form is isolated then it will need to be converted to the corresponding compound of formula (III) (hydroquinones). It is well-known in the art that benzoquinones can be chemically converted to hydroquinones (reduction) and vice versa (oxidation). This can be applied to the ansamycin natural products, as described above, such that where the benzoquinone form occurs naturally, the hydroquinone can be synthesised by a variety of methods. As an example (but not by way of limitation) this may be achieved in organic media with a source of hydride, such as but not limited to, LiAlH₄ or SnCl₂—HCl. Alternatively this transformation may be mediated by dissolving the benzoquinone form of the ansamycin in organic media and then washing with an aqueous solution of a reducing agent, such as, but not limited to, sodium hydrosulfite (Na₂S₂O₄ or sodium thionite). Preferably, this transformation is carried out by dissolving macbecin or geldanamycin in ethyl acetate and mixing this solution vigorously with an aqueous solution of sodium hydrosulfite (Muroi et al., 1980). The resultant organic solution can then be washed with water, dried and the solvent removed under reduced pressure to yield an almost quantitative amount of 18,21-dihydromacbecin or 18,21-dihydrogeldanamycin respectively.

Compounds of formula (IIB) and (IIC) such as those derived from geldanamycin may or do contain a secondary hydroxyl group at the C-11 position. In order to derivatise these compounds at the C-18 hydroxyl exclusively it may be necessary to first modify the C-11 hydroxyl. Additionally, compounds derivatised at the C-11 position in combination with derivatisation at C-18 or C-21 are specifically contemplated as compounds of the invention in their own right. Described below are methods for accomplishing this for geldanamycin, but a person of skill in the art will appreciate that these can equally be applied to other compounds of formula (IIB) and (IIC), (IIE) and (IIF) for which the parent compound contains an OH group at C-11.

For example, the C-11 hydroxyl could be oxidised to a ketone, by one of many standard protocols for oxidising secondary alcohols to a ketone, such as but not limited to, Swern oxidation, Dess-Martin periodination, tetrapropylammonium peruruthenate (TPAP), Jones' reagent, pyridinium chlorochromate (Corey's reagent) or pyridinium dichromate. With 11-oxogeldanamycin in hand the benzoquinone may now be reduced to the hydroquinone, as described above. It will be appreciated by a person of skill in the art that care must be taken to not concomitantly reduce out the newly formed C-11 ketone, in other words the reducing agent selected should be chemoselective for the benzoquinone system over the C-11 carbonyl, such chemoselective agents are known to a person of skill in the art, an example of a suitable agent (without limitation) is sodium hydrosulfite, unsuitable agents include, without limitation LiAlH₄. 11-oxo-18,21-dihydrogeldanamycin can then be used as a template for further derivatisation into a compound of Formula I, as described below.

Protecting groups may, if desired, be generally employed in the synthesis of compounds of the invention and intermediate compounds as would be understood by a person skilled in the art.

Hydroquinone ansamycins, as shown as Formula (III) converted if required from their benzoquinone forms, may be used as templates for further semi-synthesis (i.e. process (a)).

Other compounds embraced by the invention may be prepared by methods described herein and/or by methods known to a skilled person.

Compounds of formula (H) may be produced by methods known to a person of skill in the art. One suitable method for the production of compounds of formula H is illustrated below, but it will be appreciated that other, equally suitable, methods of production may be employed.

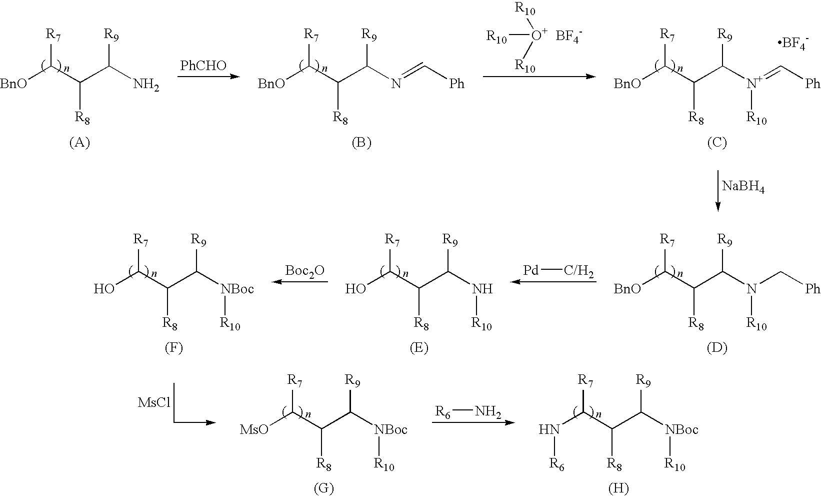

Therefore, in one embodiment, a variety of self-cleaving and water solubilizing side-chains (R₅) can be introduced at the 18 and/or 21-position of 18,21-dihydromacbecin. This may be achieved by treating a mono N-protected diamine of variable chain length and substitution pattern (H) with an intermediate 4-nitrophenyl carbonate analogue of macbecin (e.g. a compound of formula (II) above).

A primary amino alkyl benzyl ether (A), derived from protection group manipulation of an amino alcohol, is reacted with benzaldehyde to give the Schiff base (B). The imine formed is treated with an alkylating agent e.g. a trialkyloxonium tetrafluoroborate to introduce R₁₀ and thus generate an iminium salt (C). Hydride reduction to the benzylamine (D) and subsequent di-debenzylation via catalytic hydrogenolysis provides secondary amines of type (E). N-Boc protection to (F) is followed by hydroxyl activation to a suitable leaving group e.g. mesylate (G). The Boc group of compounds (F), (G) and (H) in the diagram may be replaced by a different protecting group if desired by treating compound (E) with an alternative protecting reagent. A preferred alternative protecting group is trityl.

In process (c), salt formation and exchange may be performed by conventional methods known to a person of skill in the art. Interconversions of compounds of formula (I) may be performed by known processes for example hydroxy and keto groups may be interconverted by oxidation/reduction as described elsewhere herein.

In processes (a), (b) and (d), examples of protecting groups and the means for their removal can be found in T W Greene “Protective Groups in Organic Synthesis” (J Wiley and Sons, 1991). Suitable hydroxyl protecting groups include alkyl (e.g. methyl), acetal (e.g. acetonide) and acyl (e.g. acetyl or benzoyl) which may be removed by hydrolysis, and arylalkyl (e.g. benzyl) which may be removed by catalytic hydrogenolysis, or silyl ether, which may be removed by acidic hydrolysis or fluoride ion assisted cleavage. Suitable amine protecting groups include sulphonyl (e.g. tosyl), acyl (e.g. benzyloxycarbonyl or t-butoxycarbonyl) and arylalkyl (e.g. benzyl) which may be removed by hydrolysis or hydrogenolysis as appropriate.

Compound of formula (I) in which the C18 R₅ is H may be prepared by deprotecting compounds of formula (IVA)-(IVC) (collectively known as “compounds of formula (IV)”):

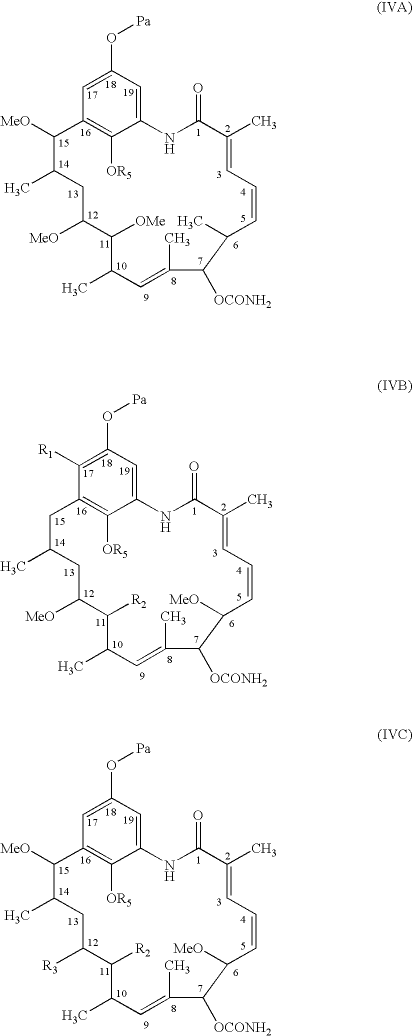

wherein R₁, R₂, R₃ and R₅ are as defined above (save that R₅ does not represent H) and Pa represents a protecting group, or a further protected derivative thereof. Exemplary hydroxyl protecting groups Pa include those mentioned above, especially silyl ethers. Deprotection by removal of the silyl group may be achieved by hydrolysis under conventional conditions.

Compounds of formula (IV) or a further protected derivative thereof may be prepared by reacting a compound of formula (VA)-(VC) (collectively known as “compounds of formula (V)”):

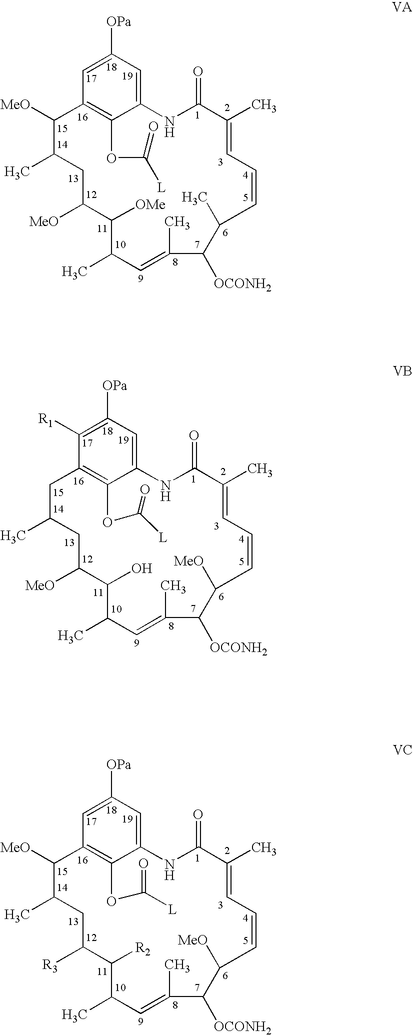

wherein L is a leaving group

or a protected derivative thereof, with a compound of formula (H).

Suitable conditions include those given above for processes (a) and (b).

Compounds of formula (V) or a protected derivative thereof may be prepared by reacting a compound of formula (VIA)-(VIC) (collectively known as “compounds of formula (VI)”):

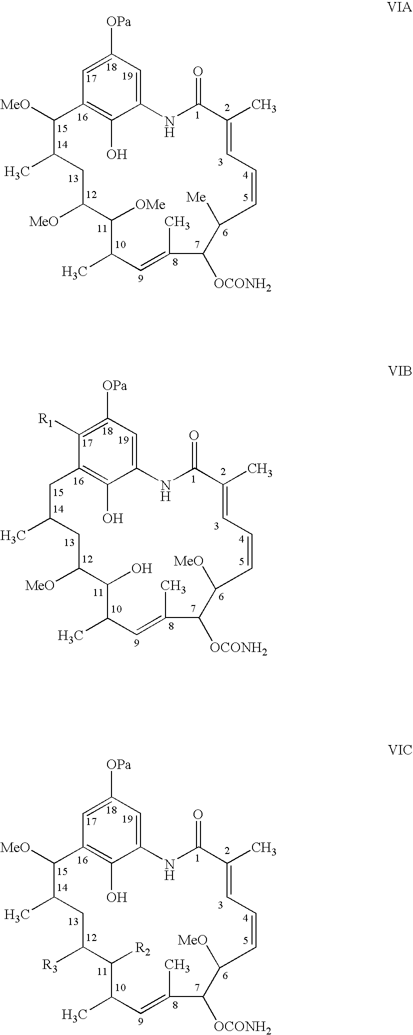

or a protected derivative thereof, with a compound of formula (J).

Suitable conditions include those given above for the reaction of compounds of formula (III) with compound of formula (J).

Compounds of formula (VI) or a protected derivative thereof may be prepared by protecting compounds of formula (III). When Pa represent a silyl ether, protection may be achieved by treating the compound of formula (III) with trialkylsilyl chloride or trialkylsilyl triflate in the presence of base.

Other compounds of the invention may be prepared by methods known per se or by methods analogous to those described above.

The compounds of the invention are useful directly, and as templates for further semi-synthesis or bioconversion, to produce compounds useful as anticancer agents. Methods for the semi-synthetic derivatisation of ansamycins such as geldanamycin and related compounds are well known in the art and include (but are not limited to) those modifications described in e.g. WO 03/013430, WO 02/079167, WO 03/066005.

In particular, the compounds of the invention have utility as pro-drugs, they may be chemically cleaved to produce the active parent compound. Cleavage assays to assess the rate of cleavage are known in the art and are

The above structures of intermediates may be subject to tautomerisation and where a representative tautomer is illustrated it will be understood that and all tautomers for example keto compounds where enol compounds are illustrated and vice versa are intended to be referred to.

Novel compounds of formula (II), (III), (IV), (V) and (VI) and protected derivatives thereof are also claimed as an aspect of the invention.

The invention additionally provides for the use of a compound of the invention in the treatment of cancer or B-cell malignancies. It also provides a compound of the invention for use in the treatment of cancer or B-cell malignancies. It also provides a method of treatment of cancer or B-cell malignancies which comprises administering to a patient an effective amount of a compound of the invention. It also provides the use of a compound of the invention in the preparation of a medicament for the treatment of cancer or B-cell malignancies.

Ansamycins are also known to have utilities in the treatment of other conditions, including, but not limited to, the treatment of cardiac arrest and stroke (U.S. Pat. No. 6,174,875, WO 99/51223), the treatment of fibrogenic disorders (WO 02/02123), the treatment or prevention of restenosis (WO 03/079936), the treatment or prevention of diseases associated with protein aggregation and amyloid function (WO 02/094259), the treatment of peripheral nerve damage and the promotion of nerve regeneration (WO 01/03692, U.S. Pat. No. 6,641,810, EP 1 024 806, US 2002/0086015, U.S. Pat. No. 6,210,974, WO 99/21552, U.S. Pat. No. 5,968,921) and the inhibition of angiogenesis (WO 04/000307). The uses and methods involving the compounds of the invention also extend to these other indications.

In a preferred embodiment, the present invention provides compounds with utility in the treatment of cancer. One skilled in the art would be able by routine experimentation to determine the ability of these compounds to inhibit tumour cell growth, (see Tian et al., 2004; Hu et al. 2004; Dengler et al, 1995).

The present invention also provides a pharmaceutical composition comprising an ansamycin derivative, or a pharmaceutically acceptable salt thereof, together with a pharmaceutically acceptable carrier.

The existing ansamycin HSP90 inhibitors that are or have been in clinical trials, such as geldanamycin, 17-AAG and 17-DMAG have poor pharmacological profiles, poor water solubility and poor bioavailability. The present invention provides ansamycin derivatives which have improved properties such as solubility and/or bioavailability. A person of skill in the art will be able to readily determine the solubility of a given compound of the invention using standard methods. A representative method is shown in the examples herein.

Additionally, a person of skill in the art will be able to determine the pharmacokinetics and bioavailability of a compound of the invention using in vivo and in vitro methods known to a person of skill in the art, including but not limited to those described below and in Egorin M J et al., (2002). The bioavailability of a compound is determined by a number of factors, (e.g. water solubility, rate of absorption in the gut, the extent of protein binding and metabolism) each of which may be determined by in vitro tests as described below, it will be appreciated by a person of skill in the art that an improvement in one or more of these factors will lead to an improvement in the bioavailability of a compound. Alternatively, the bioavailability of a compound may be measured using in vivo methods as described in more detail below.

### In Vitro Assays

### a) Caco-2 Permeation Assay

Confluent Caco-2 cells (Li, A. P., 1992; Grass, G. M., et al., 1992, Volpe, D. A., et al., 2001) in a 24 well Corning Costar Transwell format are used to establish the permeability and efflux rate of compounds using methods as described herein, suitable formats include those provided by In Vitro Technologies Inc. Baltimore, Md., USA. In a suitable format the apical chamber contains 0.15 mL HBBS pH 7.4, 1% DMSO, 0.1 mM Lucifer Yellow and the basal chamber contains 0.6 mL HBBS pH 7.4, 1% DMSO. Controls and test assays are incubated at 37° C. in a humidified incubator, shaken at 130 rpm. Lucifer Yellow is able to permeate via the paracellular route only (i.e. between the tight junctions), a high Apparent Permeability (Pₐₚₚ) for Lucifer Yellow indicates cellular damage during assay and all such wells are rejected. Suitable reference controls in addition to the parent compound include propranolol, which has good passive permeation with no known transporter effects and acebutalol, which has poor passive permeation attenuated by active efflux by P-glycoprotein.

Compounds are tested in a uni- and bi-directional format by applying compound to the apical or basal chamber (at 0.01 mM). Compounds in the apical or basal chambers are analysed by LC-MS. Results are expressed as Apparent Permeability, Pₐₚₚ, (nm/s) and as the Flux Ratio (A to B versus B to A).

$Papp ( nm \text{/} s ) = \frac{Volume Acceptor}{Area \times [ donor ]} \times \frac{Δ [ acceptor ]}{Δtime}$
Volume Acceptor: 0.6 mL (A > B) and 0.15 mL (B > A)
Area of monolayer: 0.33 cm2
Δtime: 60 min

A positive value for the Flux Ratio indicates active efflux from the apical surface of the cells. Therefore, improved bioavailability is shown in the above assay by an increased Pₐₚₚ and/or a decreased flux ratio for the compound of the invention relative to its parent molecule.

### b) Human Liver Microsomal (HLM) Stability Assay

Increased metabolic stability is also associated with improved bioavailability, this may be determined using a HLM assay for example as described below. Liver homogenates provide a measure of a compounds inherent vulnerability to Phase I (oxidative) enzymes, including CYP450s (e.g. CYP2C8, CYP2D6, CYP1A, CYP3A4, CYP2E1), esterases, amidases and flavin monooxygenases (FMOs).

The half life (T½) of compounds can be determined, on exposure to Human Liver Microsomes, by monitoring their disappearance over time by LC-MS. Compounds at 0.001 mM are incubated at for 40 min at 37° C., 0.1 M Tris-HCl, pH 7.4 with a human microsomal sub-cellular fraction of liver at 0.25 mg/mL protein and saturating levels of NADPH as co-factor. At timed intervals, acetonitrile is added to test samples to precipitate protein and stop metabolism. Samples are centrifuged and analysed for parent compound.

Improved bioavailability is shown in the above assay by an increased T½ relative to the parent compound.

### In Vivo Assays

In vivo assays may also be used to measure the bioavailability of a compound. Generally, a compound is administered to a test animal (e.g. mouse or rat) both intraperitoneally (i.p.) or intravenously (i.v.) and orally (p.o.) and blood samples are taken at regular intervals to examine how the plasma concentration of the drug varies over time. The time course of plasma concentration over time can be used to calculate the absolute bioavailability of the compound as a percentage using standard models. An example of a typical protocol is described below. Mice are dosed with 1, 10, or 75 mg/kg of the compound of the invention or the parent compound i.p. i.v. or p.o. Blood samples are taken at 5, 10, 15, 30, 45, 60, 90, 120, 180, 240, 360, 420 and 2880 minutes and the concentration of the compound of the invention or parent compound in the sample is determined via HPLC. The time-course of plasma concentrations can then be used to derive key parameters such as the area under the plasma concentration-time curve (AUC—which is directly proportional to the total amount of unchanged drug that reaches the systemic circulation), the maximum (peak) plasma drug concentration, the time at which maximum plasma drug concentration occurs (peak time), additional factors which are used in the accurate determination of bioavailability include: the compound's terminal half life, total body clearance, steady-state volume of distribution and F %. These parameters are then analysed by non-compartmental or compartmental methods to give a calculated percentage bioavailability, for an example of this type of method see Egorin et al 2002, and references therein.

The aforementioned compounds of the invention or a formulation thereof may be administered by any conventional method for example but without limitation they may be administered parenterally (including intravenous administration), orally, topically (including buccal, sublingual or transdermal), via a medical device (e.g. a stent), by inhalation, or via injection (subcutaneous or intramuscular). The treatment may consist of a single dose or a plurality of doses over a period of time.

Whilst it is possible for a compound of the invention to be administered alone, it is preferable to present it as a pharmaceutical formulation, together with one or more acceptable carriers. Thus there is provided a pharmaceutical composition comprising a compound of the invention together with one or more pharmaceutically acceptable diluents or carriers. The diluents(s) or carrier(s) must be “acceptable” in the sense of being compatible with the compound of the invention and not deleterious to the recipients thereof. Examples of suitable carriers are described in more detail below.

The compounds of the invention may be administered alone or in combination with other therapeutic agents. Co-administration of two (or more) agents may allow for significantly lower doses of each to be used, thereby reducing the side effects seen. There is also provided a pharmaceutical composition comprising a compound of the invention and a further therapeutic agent together with one or more pharmaceutically acceptable diluents or carriers.

In a further aspect, the present invention provides for the use of a compound of the invention in combination therapy with a second agent for the treatment of cancer or B-cell malignancies.

In one embodiment, a compound of the invention is co-administered with another therapeutic agent for the treatment of cancer or B-cell malignancies preferred agents include, but are not limited to, methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Iressa®, Tarceva™, Erbitux™), VEGF inhibitors (e.g. Avastin™), proteasome inhibitors (e.g. Velcade™) or Glivec®. Additionally, a compound of the invention may be administered in combination with other therapies including, but not limited to, radiotherapy or surgery.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

It should be understood that in addition to the ingredients particularly mentioned above the formulations of this invention may include other agents conventional in the art having regard to the type of formulation in question, for example those suitable for oral administration may include flavouring agents.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The compounds of the invention may also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The dosage to be administered of a compound of the invention will vary according to the particular compound, the disease involved, the subject, and the nature and severity of the disease and the physical condition of the subject, and the selected route of administration. The appropriate dosage can be readily determined by a person skilled in the art.

The compositions may contain from 0.1% by weight, preferably from 5-60%, more preferably from 10-30% by weight, of a compound of invention, depending on the method of administration.

It will be recognized by one of skill in the art that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

### Examples

### General Methods

### Fermentation of Cultures

Conditions used for growing the bacterial strains Actinosynnema pretiosum subsp. pretiosum ATCC 31280 (U.S. Pat. No. 4,315,989) and Actinosynnema mirum DSM 43827 (KCC A-0225, Watanabe et al., 1982) are described in the patents U.S. Pat. No. 4,315,989 and U.S. Pat. No. 4,187,292. Both strains may be grown on ISP2 agar (Shirling, E. B. and Gottlieb, D. (1966) International Journal of Systematic Bacteriology 16:313-340) at 28° C. for 2-3 days and used to inoculate seed medium (20 parts of glucose, 30 parts of soluble starch, 10 parts of corn steep liquor, 10 parts of soybean flour, 5 parts of peptone, 3 parts of sodium chloride and 5 parts of calcium carbonate/water 1000 parts by volume, pH 7.0, see U.S. Pat. No. 4,315,989 and U.S. Pat. No. 4,187,292). The inoculated seed medium is incubated shaking at 28° C. for 48 h. For production of macbecin and 18,21-dihydromacbecin the fermentation medium (5% of glycerol, 2% of corn steep liquor, 2% of yeast extract, 2% of KH₂PO₄, 0.5% of MgCl₂ and 0.1% of CaCO₃, pH6.5, see U.S. Pat. No. 4,315,989 and U.S. Pat. No. 4,187,292) is initially incubated at 28° C. for 24 h followed by an incubation period at 26° C. for four to six days. The culture is then harvested for extraction.

### Extraction of Culture Broths for LCMS Analysis

Culture broth (1 mL) and ethyl acetate (1 mL) is added and mixed for 15-30 min followed by centrifugation for 10 min. 0.5 mL of the organic layer is collected, evaporated to dryness and then re-dissolved in 0.25 mL of methanol.

### LCMS Analysis Procedure for Fermentation Broth Analysis and in Vivo Transformation Studies

LCMS is performed on an integrated Agilent HP1100 HPLC system in combination with a Bruker Daltonics Esquire 3000+ electrospray mass spectrometer operating in positive ion mode. Chromatography was achieved over a Phenomenex Hyperclone column (C₁₈ BDS, 3 u, 150×4.6 mm) eluting over 11 min at a flow rate of 1 mL/min with a linear gradient from acetonitrile/water (40/60) to acetonitrile/water (80/20).

### Synthesis

All reactions are conducted under anhydrous conditions unless stated otherwise, in oven dried glassware that is cooled under vacuum, using dried solvents. Reactions are monitored by LC-UV-MS, on an Agilent 1100 HPLC coupled to a Bruker Daltonics Esquire3000 electrospray mass spectrometer, switching between positive ion and negative ion modes for alternate scans. Chromatography was achieved over a Phenomenex Hyperclone column, BDS C₁₈ 3 u (150×4.6 mm), with a linear gradient of acetonitrile:water (40:60 to 100) over 11 min at 1 mL/min.

### Water Solubility Assay

Kinetic Measurements:

Stock solutions of the compounds (10 mM) in DMSO were prepared. Aliquots (0.01 mL) of each were made up to 0.5 mL with either PBS solution or DMSO. The resulting 0.2 mM solutions were shaken for at room temperature on an IKA® vibrax VXR shaker.

After shaking the resulting solutions or suspensions were transferred to 2 mL Eppendorf tubes and centrifuged for 30 minutes at 13200 rpm. Aliquots of the supernatant fluid were then analysed by an Agilent 1100 HPLC coupled to a Bruker Daltonics Esquire3000 electrospray mass spectrometer (for details see specific examples herein). Chromatography was achieved over a Phenomenex Hyperclone column, BDS C₁₈ 3 u (150×4.6 mm), with a linear gradient of acetonitrile:water (40:60 to 100) over 11 min at 1 mL/min. UV absorbance was monitored at λ=258 and 280 nm.

All analyses were performed in triplicate and the solubilities of individual compounds calculated by comparing their solubility in PBS with an assumed solubility of 100% in DMSO at 0.2 mM.

Thermodynamic Measurements:

The appropriate amounts of compound to make solutions of the compounds at 2, 5, 10 and or 20 mM were mixed with the appropriate amounts of 5% glucose and with DMSO in brown glass vials and shaken at room temperature on an IKA® vibrax VXR shaker. After 6 hours the resulting suspensions/solutions were centrifuged for 20 min at 13200 rpm.

Aliquots of the supernatant fluid are then analysed by an Agilent 1100 HPLC coupled to a Bruker Daltonics Esquire3000 electrospray mass spectrometer (for details see specific examples herein). Chromatography was achieved over a Phenomenex Hyperclone column, BDS C₁₈ 3 u (150×4.6 mm), with a linear gradient of acetonitrile:water (40:60 to 100) over 11 min at 1 mL/min. UV absorbance was monitored at λ=258 and 280 nm.

All analyses were performed in triplicate and the solubilities of individual compounds calculated by comparing their solubility in 5% glucose with an assumed solubility of 100% in DMSO.

Cleavage Assays for Pro-Drugs of 18,21-dihydromacbecin

Assays to determine the rate of cleavage of the pro-drugs into their parent compounds were performed as described herein. These were carried out in plasma, blood or buffer. Mixed mouse, human or mixed rat plasma containing EDTA or heparin as an anti-coagulant was used or whole defibrinated rabbit blood (mouse and rat plasma and rabbit blood were obtained from Harlan-Sera Labs, human plasma was obtained from Biopredic International).

If using plasma the following protocol was used: The plasma was thawed at 37° C. in a water bath. Plasma was dispensed into individual 20 mL tubes for each test compound allowing 0.5 mL plasma for each time point to be analysed. A tube containing plasma was also included as a control. These tubes were incubated for 15 minutes at 37° C.

Each test compound was reconstituted in DMSO or other appropriate solvent. The plasma was removed from the water bath and the compound was added to the plasma to give a final concentration of 0.001-0.010 mg/mL keeping the final concentration of solvent below 5%. In the case of the control, an equivalent volume of DMSO or other solvent was added. Immediately after the addition of compound a 0.4 mL sample was taken from the tube, transferred to a 2 mL microfuge tube. The plasma was promptly returned to the 37° C. water bath. 0.4 mL samples were taken at regular time points (e.g. after 30 min and then hourly) and were immediately frozen at −80° C.

The stability of the test compounds in phosphate-buffered saline (PBS) of specific pH over the course of each experiment was also analysed using the same methodology.

After the final time point was sampled, the samples were extracted as follows. For each time point sample an Oasis MCX (3 cc/60 mg) or Oasis HLB (333/60 mg) cartridge was conditioned and equilibrated with 2 mL methanol followed by 2 mL water. 1.5 mL water and an internal standard (IS) at 0.002 mg/mL were added to each 0.4 mL plasma sample. This was loaded to the cartridge. The cartridge was washed with 2 mL water and the analytes were eluted in 2 ml MeOH. This extract was analysed without further treatment by LC/MS using the conditions described below.

If using whole blood such as defibrinated whole rabbit blood the following protocol was used: The blood was thawed at 37° C. in a water bath. Blood was dispensed into individual 20 mL tubes for each test compound allowing 0.5 mL for each time point to be analysed. A tube containing blood was also included as a control. These tubes were incubated for 15 minutes at 37° C.

Each test compound was reconstituted in DMSO or other appropriate solvent. The blood was removed from the water bath and the compound was added to give a final concentration of 0.001-0.010 mg/mL keeping the final concentration of solvent below 5%. In the case of the control, an equivalent volume of DMSO or other solvent was added. Immediately after the addition of compound a 0.4 mL sample was taken from the tube, transferred to a 2 mL microfuge tube. The plasma was promptly returned to the 37° C. water bath. 0.4 mL samples were taken at regular time points (e.g. after 30 min and then hourly) and were immediately frozen at −80° C. To each 0.4 mL sample in its 2 mL microfuge tube was added 1.5 mL of 0.1 M potassium dihydrogen phosphate, this was inverted to mix. A stable internal standard (IS) in acetonitrile was added at 0.002 mg/mL. This was agitated for 5 min on an IKA shaker at 1500 rpm. Centrifugation at 5000 rpm for 5 min lead to a dense pellet made up of red blood cells and precipitated protein, and a pink/red supernatant. An Oasis HLB (333/60 mg) cartridge was equilibrated with 2 mL of methanol followed by 2 mL of water. 1.5 mL of the supernatant was transferred to the cartridge and allowed to flow through with application of mild positive pressure when necessary. Each cartridge was dried by flashing it with a volume of air from a syringe and analytes eluted with 1 mL of acetonitrile. 0.01 mL of 10% formic acid was added to the eluant which was then transferred to an HPLC vial for analysis.

The different LCMS analytical procedures used to measure the cleavage rates were:

Method A

Injection volume: 0.03 mL. HPLC was performed on a Phenomenex Hyperclone 3 micron BDS C18 column, 150 mm×4.60 mm, running a mobile phase of:

| | |
|---|---|
| Mobile phase A: | 0.1% Formic acid in water |
| Mobile phase B: | 0.1% Formic acid in acetonitrile |
| Flow rate: | 1 mL/minute. |

The HPLC conditions were: 30% B for 2 min followed by a linear gradient to 100% B over a period of 14 min and an isocratic period of 4 min at 100% B. The analytes were detected by UV absorbance at 255 nm and mass spectrometry using a Bruker Daltonics Esquire 3000+ mass spectrometer coupled to the HPLC. The analytes were quantified based on the extracted ion chromatogram. To ensure a reliable quantification, the linear response of the mass spectrometer was checked for the concentration range of interest.

Method B

Injection volume: 0.030 mL. HPLC was performed on a Waters Symmetry C8 3.5 micron column, 50 mm×2.1 mm, running a mobile phase of:

| | |
|---|---|
| Mobile phase A: | 0.1% Formic acid in water |
| Mobile phase B: | 0.1% Formic acid in acetonitrile |
| Flow rate: | 1 mL/minute. |

The HPLC conditions were: 10% B for 1 min followed by a linear gradient to 100% B over a period of 7 min and an isocratic period of 2 min at 100% B. The analytes were detected by UV absorbance at 255 nm and mass spectrometry using a Bruker Daltonics Esquire 3000+ mass spectrometer coupled to the HPLC. The analytes were quantified based on the extracted ion chromatogram. To ensure a reliable quantification, the linear response of the mass spectrometer was checked for the concentration range of interest.

Method C

Injection volume: 0.02 mL. HPLC was performed on a Phenomenex Hyperclone 3 micron BDS C18 column, 150 mm×4.60 mm, running a mobile phase of:

| | |
|---|---|
| Mobile phase A: | 0.1% Formic acid in water |
| Mobile phase B: | 0.1% Formic acid in acetonitrile |
| Flow rate: | 1 mL/minute. |

The HPLC conditions were: 10% B for 1 min followed by a linear gradient to 100% B over a period of 7 min and an isocratic period of 2 min at 100% B. The analytes were detected by UV absorbance at 255 nm and mass spectrometry using a Bruker Daltonics Esquire 3000+ mass spectrometer coupled to the HPLC. The analytes were quantified based on the extracted ion chromatogram. To ensure a reliable quantification, the linear response of the mass spectrometer was checked for the concentration range of interest.

### In Vitro Bioassay for Anticancer Activity

In vitro evaluation of compounds for anticancer activity in a panel of 12 human tumour cell lines in a monolayer proliferation assay may be carried out at the Oncotest Testing Facility, Institute for Experimental Oncology, Oncotest GmbH, Freiburg. The characteristics of the 12 selected cell lines are summarised in Table 3.

TABLE 3
Test cell lines

| # | Cell line | Characteristics |
|---|---|---|
| 1 | MCF-7 | Breast, NCI standard |
| 2 | MDA-MB-231 | Breast - PTEN positive, resistant to 17-AAG |
| 3 | MDA-MB-468 | Breast - PTEN negative, resistant to 17-AAG |
| 4 | NCI-H460 | Lung, NCI standard |
| 5 | SF-268 | CNS, NCI standard |
| 6 | OVCAR-3 | Ovarian - p85 mutated. AKT amplified. |
| 7 | A498 | Renal, high MDR expression, |
| 8 | GXF 251L | Gastric |
| 9 | MEXF 394NL | Melanoma |
| 10 | UXF 1138L | Uterus |
| 11 | LNCAP | Prostate - PTEN negative |
| 12 | DU145 | Prostate - PTEN positive |

The Oncotest cell lines were established from human tumor xenografts as described by Roth et al., (1999). The origin of the donor xenografts is described by Fiebig et al., (1999). Other cell lines were either obtained from the NCI (H460, SF-268, OVCAR-3, DU145, MDA-MB-231, MDA-MB-468) or purchased from DSMZ, Braunschweig, Germany (LNCAP).

All cell lines, unless otherwise specified, were grown at 37° C. in a humidified atmosphere (95% air, 5% CO₂) in a ‘ready-mix’ medium containing RPMI 1640 medium, 10% fetal calf serum, and 0.1 mg/mL gentamicin (PAA, Cölbe, Germany).

### Monolayer Assay—Brief Description of Protocol:

A modified propidium iodide assay was used to assess the effects of the test compound(s) on the growth of twelve human tumour cell lines (Dengler et al., (1995)).

Briefly, cells were harvested from exponential phase cultures by trypsinization, counted and plated in 96 well flat-bottomed microtitre plates at a cell density dependent on the cell line (5-10.000 viable cells/well). After 24 h recovery to allow the cells to resume exponential growth, 0.010 mL of culture medium (6 control wells per plate) or culture medium containing macbecin was added to the wells. Each concentration was plated in triplicate. Compounds were applied in two concentrations (0.001 mM and 0.01 mM). Following 4 days of continuous exposure, cell culture medium with or without test compound is replaced by 0.2 mL of an aqueous propidium iodide (PI) solution (7 mg/L). To measure the proportion of living cells, cells are permeabilized by freezing the plates. After thawing the plates, fluorescence was measured using the Cytofluor 4000 microplate reader (excitation 530 nm, emission 620 nm), giving a direct relationship to the total number of viable cells.

Growth inhibition is expressed as treated/control×100 (% T/C).

### In Vivo Evaluation of Antitumor Efficacy—Brief Description of Protocol:

PRXF DU-145 is a prostate cancer cell line initially isolated from a metastatic central nervous system lesion of a 69-year-old man with prostate carcinoma. PRXF DU-145 cell suspensions were injected subcutaneously into nude mice and the resulting xenografts were passaged in nude mice until establishment of a stable growth pattern.

Tumour fragments were obtained from xenografts in serial passage in nude mice. After removal of tumours from donor mice, they were cut into fragments (1-2 mm diameter) and placed in RPMI 1640 culture medium (RPMI 1640 medium with 25 mM HEPES buffer with L-Glutamine, Gibco, catalogue #52400-025) until subcutaneous implantation. Recipient mice were anaesthetized by inhalation of isoflurane. For bilateral implantation a small incision was made in the skin of the back. Tumour fragments were transplanted with tweezers.

At randomization, tumour bearing animals were stratified into treatment and control groups according to tumour volume, using “Lindner's Randomization Tables”. Only animals carrying a tumour of appropriate size (mean tumor diameter: 6-8 mm, minimum acceptable tumor diameter: 5 mm) were considered for randomization. Mice were randomized when a maximum number of mice qualified for randomization. The day of randomization was designated as Day 0. Day 0 was also the first day of dosing. Tumour growth was then compared between mice given different dosing regimes. Mice were monitored daily, with the observation period lasting 42 Days.

The tumour volume was determined by two-dimensional measurement with a caliper on the day of randomization (Day 0) and then twice weekly. Tumour volumes were calculated according to the formula: (a×b2)×0.5 where a represents the largest and b the perpendicular tumor diameter.

Antitumour activity was evaluated as maximum tumour volume inhibition versus the vehicle control group.

Rel. volumes of individual tumors (RTVs) were calculated by dividing the individual tumor volume on Day X (Tx) by the individual tumor volume on Day 0 (T0) multiplied by 100%.

$$Ind . RTV = \frac{Tx}{T 0} \times 100 \%$$

Group tumor volumes were expressed as the median RTV of all tumours in a group (group median RTV). Group median RTV values were used for drawing growth curves and for treatment evaluation.

For each group, tumour inhibition on a particular day (T/C in %) was calculated from the ratio of the median RTV values of the respective test group and the vehicle control group, multiplied by 100%.

$$T \text{/} C ( Day x ) = \frac{\begin{matrix}Median relative tumor volume \\ of the test group Day x\end{matrix}}{\begin{matrix}Median relative tumor volume \\ of the control group Day x\end{matrix}} \times 100 \%$$

The minimum (or optimum) T/C % value recorded for a particular test group during an experiment represents the maximum antitumour activity for the respective treatment.

For the evaluation of the statistical significance of tumour inhibition, the U-test by Mann-Whitney-Wilcoxon was performed. The test compares the ranking of individual tumours according to relative volume in the vehicle control group on the one hand and in the test group of interest on the other. By convention, p-values<0.05 indicate significance of tumour inhibition.

Intermediate 1: Production of Macbecin Using Actinosynnema Mirum DSM 43827 & Actinosynnema Pretiosum Subsp. Pretiosum ATCC 31280 in Falcon Tubes

Falcon tubes containing 10 mL of seed medium were inoculated with an agar plug cut from a dense A. mirum lawn grown on ISP2 agar and incubated as described in General Methods. The seed culture (0.5 mL) was used for inoculation of 10 mL of production medium followed by incubation and extraction as described in the General Methods section. Analysis by LCMS indicated the presence of macbecin ([M+Na]⁺, m/z=581.3) which eluted at 9.5 min.

### Intermediate 1 (Alternative Preparation): Production of Macbecin Using Actinosynnema Mirum DSM 43827 in a 15 Litre Fermenter

Five 2 L conical shaking flasks were prepared with 300 mL of seed medium each and inoculated with 15 agar plugs per flask cut from a densely grown lawn of A. mirum. The cultures were shaken at 28° C. for 48 h. These seed cultures were used to inoculate (10% inoculum) a fermentation vessel containing 15 L of production medium. The fermentation was carried out with an impeller tip speed of between 1.18 m/s and 2.75 m/s, an air flow of 1 vvm; these were heated at 28° C. for 24 h then reduced to 24° C. The pH was adjusted between pH 6.5-7 with 1 M H₂SO₄ and 1 M NaOH during the fermentation run. The baffles were tilted at 45° and impeller tip speed was adjusted according to oxygen demand (minimum dissolved oxygen was maintained at 30%). Antifoam SAG471 was added at 0.2% v/v prior to autoclaving and then as and when required during the fermentation run. The fermenter was harvested after 230 h. Analysis by LCMS indicated the presence of macbecin ([M+Na]⁺, m/z=581.3) which eluted after 9.5 min.

The fermentation broth was centrifuged for 30 min at 3500 rpm to separate the cells from the supernatant. The supernatant was then partitioned three times with an equal volume of ethyl acetate. The cell pellet was extracted twice with an equal volume of acetone. The organic fractions were combined and the solvent removed in vacuo. The resulting aqueous slurry was extracted three times with an equal volume of ethyl acetate and the combined organic fractions concentrated to yield a crude extract.

Flash Silica gel was added to the crude extract previously dissolved in acetone and the solvent then removed in vacuo. The impregnated silica was added onto an open column of flash silica pre-conditioned with hexane. This was eluted with a step gradient of hexane:ethyl acetate (100:0, 80:20, 75:25, 70:30, 50:50, 0:100) and finally ethyl acetate:methanol (50:50, 0:100). Each step fraction volume was about one litre of solvent mixture. The fractions containing macbecin were combined and evaporated to dryness. Further purification was realised by preparative reversed-phase HPLC over a Phenomenex Luna column (C₁₈, 10 micron, 250×5 u, 250×21.20 mm) eluting over 30 min with a gradient of eluant A (acetonitrile:water, 20:80) to eluant B (acetonitrile) at a flow rate of 21 mL/min. Fractions containing macbecin were combined and concentrated to yield to a yellow powder (45 mg). The structure of macbecin (in CDCl₃) was confirmed by multidimensional NMR spectroscopy using a Bruker Advance 500 MHz cryoprobe instrument, see Table 4. This was consistent with published data (Muroi et al. 1981).

TABLE 4

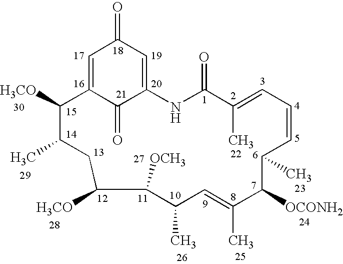

| | | 500 MHz CDCl₃ | | | | published |
|---|---|---|---|---|---|---|
| Position | | δ_{C} | δ_{H} (mult., Hz) | H-H COSY | H-C HMBC | δ_{H} (mult., Hz) |
| 1 | C═ | 169.3 | — | — | — | — |
| 2 | C═ | 133.3 | — | — | — | — |
| 3 | HC═ | 128.9 | 7.13 d 12.0) | 4, 22 | 1, 2, 5, 22 | 7.14 dd |
| 4 | HC═ | 124.2 | 6.33 td (11.7) | 3, 5, 6 | 2, 3, 5, 6 | 6.35 dt |
| 5 | HC═ | 141.3 | 5.67 dd (9.9, 7.1) | 4, 6 | 3, 23 | 5.67 dd |
| 6 | CH | 33.4 | 3.09 m | 5, 7, 23 | 4, 5, 23 | 3.10 m |
| 7 | CH | 79.3 | 5.80 bs | 6 | — | 5.78 d |
| 8 | C═ | 131.6 | — | — | — | — |
| 9 | HC═ | 127.7 | 5.25 bs | 10, 25 | | 5.30 bd |
| 10 | CH | 34.7 | 2.49 bs | 9, 11, 26 | — | 2.55 m |
| 11 | CH | 83.5 | 3.24 m | 10 | — | 3.25 dd |
| 12 | CH | 83.0 | 3.56 | 11, 13 | — | 2.95 m |
| 13 | CH₂ | 33.9 | 1.66 m | 12 | — | 1.80 m |
| 14 | CH | 33.7 | 1.50 m | 13, 29 | — | 1.58 m |
| 15 | CH | 76.9 | 4.60 bs | 17 | 16 | 4.60 dd |
| 16 | C═ | 144.8 | — | — | — | — |
| 17 | HC═ | 132.3 | 6.61 d | 15, 19 | 15, 16, 19, 21 | 6.62 dd |
| 18/21 | C═O | 187.9 or | — | — | — | — |
| 19 | HC═ | 112.9 | 7.33 d (2.0) | 17 | 17, 20, 21 | 7.33 d |
| 20 | C═ | 138.3 | — | — | — | — |
| 21/18 | C═O | 184.0 or | — | — | — | — |
| 22 | CH₃ | 12.3 | 1.99 bs | 3 | 1, 2, 3 | 2.01 d |
| 23 | CH₃ | 13.4 | 1.04 d (6.9) | 6 | 5, 6, 7 | 1.04 |
| 24 | OCO(NH₂) | 155.8 | — | — | — | — |
| 25 | CH₃ | 15.1 | 1.49 bs | 9 | 7, 8, 9 | 1.53 bs |
| 26 | CH₃ | 17.2 | 1.08 d (6.43) | 10 | 9, 10, 11 | 1.10 d |
| 27 | OCH₃ | 60.3 | 3.53 s | — | 11 | 3.56 s |
| 28 | OCH₃ | 55.6 | 3.33 s | — | 12 | 3.33(30) s |
| 29 | CH₃ | 13.2 | 0.79 (6.9) | 14 | 14, 15 | 0.8 d |
| 30 | OCH₃ | 58.3 | 3.30 s | — | 15 | 3.30(33) s |
| | NH | — | 8.89 bs | — | 1, 19, 20 | 8.87 bs |
| | NH₂ | — | 4.60 bs | — | — | 4.61 bs |

### Example 1

### Synthesis of 18-O—(N,N′-dimethylethylenediamine-N′-carbamoyl)-18,21-dihydromacbecin Hydrochloride Salt, 1 (Route 1)

### Conversion of Macbecin to 18,21-dihydromacbecin

Macbecin (107.8 mg, 0.193 mmol) was dissolved in ethyl acetate (25 mL) and treated with 96 mM sodium hydrosulfite solution (3×5 mL). On each occasion the phases were vigorously mixed in a separating funnel and the aqueous drained off. The organic layer goes from an intense yellow colour to virtually colourless. This organic layer was then washed with water (3×10 mL), before being dried with anhydrous sodium sulfate, filtered and the solvent removed under reduced pressure to yield 18,21-dihydromacbecin as an off-white glassy solid (105.0 mg, 0.187 mmol, 97% isolated yield). 18,21-dihydromacbecin was used without any further purification

LCMS: macbecin, RT=8.2 minutes ([M−H]⁻, m/z=557.5, [M+Na]⁺, m/z=581.2) UV λₘₐₓ=256 (sh) nm; 18,21-dihydromacbecin, RT=3.5 minutes ([M−H]⁻, m/z=559.5, [M+Na]⁺, m/z=583.3) UV λₘₐₓ=302 nm.

### Preparation of N-tert-Butoxycarbonyl-N,N′-dimethylethylenediamine

N,N′-dimethylethylenediamine (1.0 g, 11.3 mmol) was dissolved in anhydrous dichloromethane (10 mL) and was treated with triethylamine (1.6 mL, 11.3 mmol). The mixture was cooled to 0° C. for the addition of di-tert-butyl dicarbonate (2.5 g, 11.3 mmol). The reaction stirred for 30 min at 0° C. then 2 hours at room temperature. The reaction mixture was then washed with water (10 mL) and the aqueous layer extracted with further portions of dichloromethane (2×10 mL). The combined organic phases were dried over Na₂SO₄ and the solvent removed in vacuo. Purification by column chromatography (40:8:1, dichloromethane:methanol:aqueous ammonia) yielded (508 mg, 24%) of the desired N-tert-butoxycarbonyl-N,N′-dimethylethylenediamine as a colourless oil.

### O-Acylation of 18,21-dihydromacbecin with N-(tert-butoxycarbonyl)-N,N′-dimethylethylenediamine to Yield 18-O—[(N-tert-butoxycarbonyl)-N,N′-dimethylethylenediamine-N′-carbamoyl]-18,21-dihydromacbecin

18,21-dihydromacbecin (2.00 mg, 3.6×10⁻³ mmol) was dissolved in anhydrous degassed dichloromethane (1 mL). 4-Nitrophenylchloroformate (0.86 mg, 4.3×10⁻³ mmol) was added followed by 2,6-lutidine (2.48×10⁻³ mL, 21.4×10⁻³ mmol). The reaction mixture was heated to reflux for 4 hrs to form the intermediate carbonate. N-tert-Butoxycarbonyl-N,N′-dimethylethylenediamine (2.70 mg, 14.2×10⁻³ mmol) was added and the reaction heated to reflux for a further 2 h. The reaction mixture was washed with water (2 mL) and the crude material analysed. The data corresponded to the that for the desired 18-O—[(N-tert-butoxycarbonyl)-N,N′-dimethylethylenediamine-N′-carbamoyl]-18,21-dihydromacbecin.

LCMS: 18-O—[(N-tert-Butoxycarbonyl)-N,N′-dimethylethylenediamine-N′-carbamoyl]-18,21-dihydromacbecin, RT=6.8 minutes ([M−H]⁻, m/z=773.4), UV λₘₐₓ=266 nm.

### Deprotection of 18-O—[(N-tert-butoxycarbonyl)-N,N′-dimethylethylenediamine-N′-carbamoyl]-18,21-dihydromacbecin to Yield 18-O—(N,N′dimethylethylenediamine-N′-carbamoyl)-18,21-dihydromacbecin Hydrochloride Salt

18-O—[(N-tert-butoxycarbonyl)-N,N′-dimethylethylenediamine-N′-carbamoyl]-18,21-dihydromacbecin (20 mg, 25.8 μmol) was treated with excess 2 M HCl in diethyl ether until all the starting material was consumed. The residue was purified by tituration with diethyl ether to yield 18-O—(N,N′-dimethylethylenediamine-N′-carbamoyl)-18,21-dihydromacbecin hydrochloride salt (11 mg, 60%) as a pale yellow solid.

Direct infusion MS: 18-O—(N,N′-dimethylethylenediamine-N′-carbamoyl)-18,21-dihydromacbecin, [M−H]⁺, m/z=673.5, [M+Na]⁺, m/z=697.4, [M+NH]⁺, m/z=675.4.

### Example 2

### Synthesis of 18-O—(N,N′-dimethylethylenediamine-N′-carbamoyl)-18,21-dihydromacbecin Hydrochloride Salt, 1 (Route 2)

### Preparation of 18-O-(4-nitrophenylcarbonate)-18,21-dihydromacbecin

Macbecin II (0.30 g, 0.54 mmol) was dissolved in anhydrous dichloromethane (72 ml). To this solution was added 4-nitrophenylchloroformate as a solid (0.183 g, 0.91 mmol) followed by 2,6-lutidine (0.217 ml, 1.87 mmol). The reaction mixture was heated at reflux under argon for 5 hours at 50° C. (oil bath). The reaction was allowed to cool to ambient and washed successively with equal volumes of 1N HCl and water, dried over Na₂SO₄ and filtered, and the solvent removed under reduced pressure. The resulting material was purified over silica gel eluting with a stepped gradient of acetone in hexane (5-40% acetone, increasing in 5% increments) to yield the title compound. Isolated yield: 0.310 g (79%). NMR spectra acquired in CDCl₃ at 400 MHz were consistent with the title compound.

LCMS: RT=7.2 min ([M−H]⁻, m/z=724.0).

### Preparation of N-trityl-N,N′-dimethylethylenediamine

N,N′-dimethylethylenediamine (5.0 g, 56.7 mmol) was dissolved in dichloromethane (20 ml) under argon and then treated with trimethylamine (5.74 g, 56.7 mmol). The stirring mixture was cooled to 0° C. prior to drop wise slow addition of a solution of tritylchloride (15.82 g, 56.7 mmol) in dichloromethane (20 mL). Following complete addition of this solution the reaction mixture was stirred at 0° C. for a further 30 min, at which point the cooling bath was removed and the reaction allowed to warm up to room temperature. The mixture was stirred under argon at room temperature overnight. The resulting solution was partitioned between dichloromethane and water, and a white precipitate of the HCl salt of the product was observed. This mixture was the treated with a 10% aqueous solution of K₂CO₃ (100 mL) which resulted in dissolution of the white solid and partitioning of the two phases. The two phases were separated and the aqueous phase extracted with dichloromethane (2×200 mL). The combined organic extracts were dried over MgSO₄ and the solvent removed under reduced pressure. The residue was purified over silica gel eluting with dichloromethane:methanol:aqueous ammonia (80:5:0.5). Pure fractions were collected and the solvents removed under reduced pressure. Remaining water/ammonia was removed by addition of isopropyl alcohol followed by removal under reduced pressure (3×300 ml) and then under high vacuum to yield the title compound as a pale yellow solid. Isolated yield, 6.30 g (34%). ¹H NMR spectra acquired in CDCl₃ at 400 MHz were consistent with the title compound.

### Preparation of 18-O—(N-trityl-N,N′-dimethylethylenediamine-N′-carbamoyl)-18-21-dihydromacbecin

18-O-(4-nitrophenylcarbonate)-18,21-dihydromacbecin (0.89 g, 1.23 mmol) was dissolved in dichloromethane (40 mL). N-trityl-N,N′-dimethylethylenediamine (1.21 g, 3.68 mmol) in dichloromethane (40 mL) was then added and the solution heated at reflux for 2 h, and then overnight at room temperature. TLC showed the presence of starting material and the mixture was refluxed for a further 2 h. This was then cooled to room temperature, washed with water, dried over Na₂SO₄ and the solvent removed under reduced pressure. The resulting material was purified by chromatography over silica gel eluting with hexane:acetone (5:3) to yield a pale green solid. Isolated yield: 0.92 g (82%). NMR spectra acquired in CDCl₃ at 400 MHz were consistent with the title compound.

LCMS: RT=11.1 min ([M−H]⁻, m/z=915.6 (weak); [M−H-trityl]⁻, m/z=673.4; [M+H-trityl]⁺, m/z=675.5).

### Preparation of 18-O—(N,N′-dimethylethylenediamine-N′-carbamoyl)-18,21-dihydromacbecin Hydrochloride Salt, 1

18-O—(N-trityl-N,N′-dimethylethylenediamine-N′-carbamoyl)-18,21-dihydromacbecin (0.10 g, 0.11 mmol) was dissolved in anhydrous dichloromethane (40 mL) under argon and cooled to −5° C. using an ice/salt bath. A solution of 2 M HCl in ether (0.104 mL, 0.21 mmol) was dissolved in anhydrous dichloromethane (0.2 mL) and then added drop wise to the cooled substrate solution. The reaction was stirred for 5 min at −5° C. and then 90 min at room temperature. Hexane (40 mL) was added to precipitate the product salt and remaining starting material. The precipitate was triturated and then washed twice with cold ether to remove any starting material and other impurities. Isolated yield: 0.060 g (77%).

LCMS: RT=3.1 min ([M−H]⁻, m/z=673.5; [M+H]⁺, m/z=675.4).

### Example 3

### Synthesis of 18-O—(N-methylethylenediamine-N′-carbamoyl)-18,21-dihydromacbecin Hydrochloride Salt, 2

### Preparation of N-Trityl-N-methylethylenediamine

N-Methylethylenediamine (5.96 g, 80.41 mmol) was dissolved in dichloromethane (100 mL) under argon. The stirring mixture was cooled to 0° C. prior to drop wise slow addition of a solution of tritylchloride (6.47 g, 23.21 mmol) in dichloromethane (40 ml). Following complete addition of this solution the reaction mixture was stirred at 0° C. for a further 30 min, at which point the cooling bath was removed and the reaction allowed to warm up to room temperature. The mixture was stirred under argon at room temperature overnight. The solvent was removed from the resulting solution and ethyl acetate (200 mL) and saturated aqueous NaHCO₃ (200 mL) were added. The resulting mixture was shaken, separated, and the aqueous extracted with a further equal volume of ethyl acetate. The combined organics were dried over Na₂SO₄, filtered and the solvent removed under reduced pressure. The residue was purified over silica gel eluting with dichloromethane:methanol:aqueous ammonia (80:5:0.1). Two regioisomers were observed and isolated separately. Pure fractions of the title compound (the more polar on silica) were collected and the solvents removed under reduced pressure. Isolated yield, 1.66 g (22%). ¹H NMR spectra acquired in DMSO at 400 MHz were consistent with the title compound and allowed assignment of the correct regioisomer.

### Preparation of 18-O—(N-trityl-N-methylethylenediamine-N′-carbamoyl)-18,21-dihydromacbecin

18-O-(4-nitrophenylcarbonate)-18,21-dihydromacbecin (0.100 g, 0.14 mmol) was dissolved in dichloromethane (20 ml). N-Trityl-N-methylethylenediamine (0.131 g, 0.41 mmol) was then added and the solution heated at reflux for 2 hr, and then overnight at room temperature. The organics were washed with water (20 mL) and then dried over anhydrous Na₂SO₄, filtered and the solvent removed under reduced pressure. The material was purified by chromatography over the silica gel column eluting with hexane:acetone (7:3). Isolated yield: 0.093 g (74%). NMR spectra acquired in CDCl₃ at 400 MHz were consistent with the title compound.

LCMS: RT=11.2 min ([M−H]⁻, m/z=901.4).

### Preparation of 18-O—(N-methylethylenediamine-N′-carbamoyl)-18,21-dihydromacbecin Hydrochloride Salt, 2

18-O—(N-trityl-N-methylethylenediamine-N′-carbamoyl)-18,21-dihydromacbecin (0.093 g, 0.10 mmol) was dissolved in anhydrous dichloromethane (19.5 mL) under argon and cooled to −5° C. using an ice/salt bath. A solution of 2M HCl in ether (0.098 mL, 0.196 mmol) was then added drop wise to the cooled substrate solution. The reaction was stirred for 5 min at −5° C. and then 60 min at room temperature. Hexane (20 mL) was added to precipitate the product salt and remaining starting material. The precipitate was triturated and then washed twice with cold ether to remove any starting material and other impurities. The resulting solid was recrystallized from dichloromethane:ether. Isolated yield: 0.021 g (29%).

LCMS: 9.7 min ([M−H]⁻, 658.9).

### Example 4

### Synthesis of 18-O—(N,N′-diethylethylenediamine-N′-carbamoyl)-18,21-dihydromacbecin Hydrochloride Salt, 3

### Preparation of N-trityl-N,N′-diethylethylenediamine

N,N′-diethylethylenediamine (2.5 g, 21.5 mmol) was dissolved in dichloromethane (20 ml) under argon. The stirring mixture was cooled to 0° C. prior to drop wise slow addition of a solution of tritylchloride (2.4 g, 8.6 mmol) in dichloromethane (20 mL). Following complete addition of this solution the reaction mixture was stirred at 0° C. for a further 30 min, at which point the cooling bath was removed and the reaction allowed to warm up to room temperature. The mixture was stirred under argon at room temperature overnight. The solvent was removed from the resulting solution and ethyl acetate (200 mL) and saturated aqueous NaHCO₃ (200 mL) were added. The resulting mixture was shaken, separated, and the aqueous extracted with a further equal volume of ethyl acetate. The combined organics were dried over Na₂SO₄, filtered and the solvent removed under reduced pressure. The residue was purified over silica gel eluting with dichloromethane:methanol:aqueous ammonia (80:5:0.5). Pure fractions were collected and the solvents removed under reduced pressure. Remaining water/ammonia was removed by addition of isopropyl alcohol and subsequent removal under reduced pressure (2×100 mL), and then under high vacuum to yield the title compound as a pale yellow solid. Isolated yield: 2.60 g (84%). ¹H NMR spectra acquired in CDCl₃ at 400 MHz were consistent with the title compound.

### Preparation of 18-O—(N-trityl-N,N′-diethylethylenediamine-N′-carbamoyl)-18,21-dihydromacbecin

18-O-(4-nitrophenylcarbonate)-18,21-dihydromacbecin (0.296 g, 0.41 mmol) was dissolved in dichloromethane (15 mL). N-trityl-N,N′-diethylethylenediamine (0.438 g, 1.22 mmol) in dichloromethane (15 mL) was then added and the solution heated at reflux for 2 hr, and then overnight at room temperature. The resulting solution was washed with water, dried over Na₂SO₄ and the solvent removed under reduced pressure. The resulting material was purified by chromatography over silica gel eluting with a stepwise gradient acetone in hexane (5-25% acetone) to yield a pale yellow solid. Isolated yield: 0.23 g (59%). NMR spectra acquired in CDCl₃ at 400 MHz were consistent with the title compound.

LCMS: the material cleaved under mobile phase conditions, losing the trityl group to liberate the secondary amine, RT=4 min (broad) ([M−H]⁻, m/z=701.6; [M+H]⁺, m/z=703.6).

### Preparation of 18-O—(N,N′-diethylethylenediamine-N′-carbamoyl)-18,21-dihydromacbecin Hydrochloride Salt, 3

18-O—(N-trityl-N,N′-diethylethylenediamine-N′-carbamoyl)-18,21-dihydromacbecin (0.206 g, 0.22 mmol) was dissolved in anhydrous dichloromethane (40 mL) under argon and cooled to −5° C. using an ice/salt bath. A solution of 2M HCl in ether (0.207 mL, 0.41 mmol) was dissolved in anhydrous dichloromethane (1 mL) and then added drop wise to the cooled substrate solution. The reaction was stirred for 5 min at −5° C. and then 90 min at room temperature. Hexane (50 mL) was added to precipitate the product salt and remaining starting material. The precipitate was triturated and then washed twice with cold ether to remove any starting material and other impurities. Isolated yield: 0.090 g (55%).

LCMS: RT=3.4 min ([M−H]⁻, m/z=701.6; [M+H]⁺, m/z=703.6).

### Example 5

### Synthesis of 18-O—(N,N′-dimethyl-1,3-propanediamine-N′-carbamoyl)-18,21-dihydromacbecin Hydrochloride Salt, 4

### Preparation of N-Trityl-N,N′-dimethyl-1,3-propanediamine

N,N′-Dimethyl-1,3-propanediamine (2.50 g, 24.5 mmol) was dissolved in dichloromethane (20 mL) under argon. The stirring mixture was cooled to 0° C. prior to drop wise slow addition of a solution of tritylchloride (2.73 g, 9.80 mmol) in dichloromethane (20 mL). Following complete addition of this solution the reaction mixture was stirred at 0° C. for a further 30 min, at which point the cooling bath was removed and the reaction allowed to warm up to room temperature. The mixture was stirred under argon at room temperature overnight. The solvent was removed from the resulting solution and ethyl acetate (200 mL) and saturated aqueous NaHCO₃ (200 mL) were added. The resulting mixture was shaken, separated, and the aqueous extracted with a further equal volume of ethyl acetate. The combined organics were dried over Na₂SO₄, filtered and the solvent removed under reduced pressure. The residue was purified over silica gel eluting with dichloromethane:methanol:aqueous ammonia (80:5:0.5). Pure fractions were collected and the solvents removed under reduced pressure. Remaining water/ammonia was removed addition of ethanol (2×100 mL) and removal under reduced pressure at 50° C., and then drying under high vacuum to yield the title compound. Isolated yield, 2.60 g (76%). ¹H NMR spectra acquired in CDCl₃ at 400 MHz were consistent with the title compound.

### Preparation of 18-O—(N-trityl-N,N′-dimethyl-1,3-propanediamine-N′-carbamoyl)-18,21-dihydromacbecin

18-O-(4-nitrophenylcarbonate)-18,21-dihydromacbecin (0.161 g, 0.22 mmol) was dissolved in dichloromethane (15 mL). N-Trityl-N,N′-dimethyl-1,3-propanediamine (0.229 g, 0.67 mmol) in dichloromethane (15 mL) was then added and the solution heated at reflux for 3.5 hr, and then overnight at room temperature. The resulting solution was washed with water, dried over Na₂SO₄ and the solvent removed under reduced pressure. The resulting material was purified by chromatography over silica gel eluting with a stepwise gradient of acetone in hexane (30-50% acetone) to yield a pale yellow solid. Isolated yield: 0.080 g (37%). NMR spectra acquired in CDCl₃ at 400 MHz were consistent with the title compound.

LCMS: RT=8.3 min ([M−H-trityl]⁻, m/z=687.5; [M+H-trityl]⁺, m/z=689.5); the material also cleaved under mobile phase conditions to liberate the free amine, RT=3.2 min ([M−H-trityl]⁻, 687.5; [M+H-trityl]⁺, 689.5).

### Preparation of 18-O—(N,N′-dimethyl-1,3-propanediamine-N′-carbamoyl)-18,21-dihydromacbecin Hydrochloride Salt, 4

18-O—(N-trityl-N,N′-dimethyl-1,3-propanediamine-N′-carbamoyl)-18,21-dihydromacbecin (0.075 g, 0.08 mmol) was dissolved in anhydrous dichloromethane (8 mL) under argon and cooled to −5° C. using an ice/salt bath. A solution of 2M HCl in ether (0.079 mL, 0.16 mmol) was dissolved in anhydrous dichloromethane (0.5 mL) and then added drop wise to the cooled substrate solution. The reaction was stirred for 5 min at −5° C. and then 90 min at room temperature. Hexane (20 mL) was added to precipitate the product salt and remaining starting material. The precipitate was triturated and then washed twice with cold ether to remove any starting material and other impurities. Isolated yield: 0.038 mg (65%).

LCMS: RT=3.3 min ([M−H]⁻, m/z=687.7; [M+H]⁺, 689.7).

### Example 6

### Synthesis of 18-O—(N,N′-diisopropylethylenediamine-N′-carbamoyl)-18,21-dihydromacbecin Hydrochloride Salt, 5

### Preparation of 18-O—(N-trityl-N,N′-diisopropylethylenediamine-N′-carbamoyl)-18,21-dihydromacbecin

18-O-(4-nitrophenylcarbonate)-18,21-dihydromacbecin (0.161 g, 0.22 mmol) was dissolved in dichloromethane (15 mL). N-trityl-N,N′-diisopropylethylenediamine (0.257 g, 0.67 mmol) in dichloromethane (15 mL) was then added and the solution heated at reflux for 3.5 h, and then overnight at room temperature. The resulting solution was washed with water, dried over Na₂SO₄ and the solvent removed under reduced pressure. The resulting material was purified by chromatography over silica gel eluting with a stepwise gradient acetone in hexane (30-50% acetone) to yield a pale yellow solid. Isolated yield: 0.060 g (28%). NMR spectra acquired in CDCl₃ at 400 MHz were consistent with the title compound.

LCMS: RT=7.8 min [M−H-trityl]⁻, m/z=729.6; [M+H-trityl]⁺, m/z=731.6.; the material also cleaved under mobile phase conditions to liberate the secondary amine, RT=5.7 min ([M−H]⁻, m/z=729.6; [M+H]⁺, m/z=731.6).

### Preparation of 18-O—(N,N′-diisopropylethylenediamine-N′-carbamoyl)-18,21-dihydromacbecin Hydrochloride Salt, 5

18-O—(N-trityl-N,N′-diisopropylethylenediamine-N′-carbamoyl)-18,21-dihydromacbecin (0.055 g, 0.06 mmol) was dissolved in anhydrous dichloromethane (10 mL) under argon and cooled to −5° C. using an ice/salt bath. A solution of 2M HCl in ether (0.055 mL, 0.11 mmol) was dissolved in anhydrous dichloromethane (1 mL) and then added drop wise to the cooled substrate solution. The reaction was stirred for 5 min at −5° C. and then 90 min at room temperature. Hexane (20 mL) was added to precipitate the product salt and remaining starting material. The precipitate was triturated and then washed twice with cold ether to remove any starting material and other impurities. Isolated yield: 0.032 mg (70%).

LCMS: RT=5.5 min ([M−H]⁻, m/z=729.9; [M+H]⁺, 731.9).

### Example 7

### Cleavage Assay for Compound 1 in Human Plasma

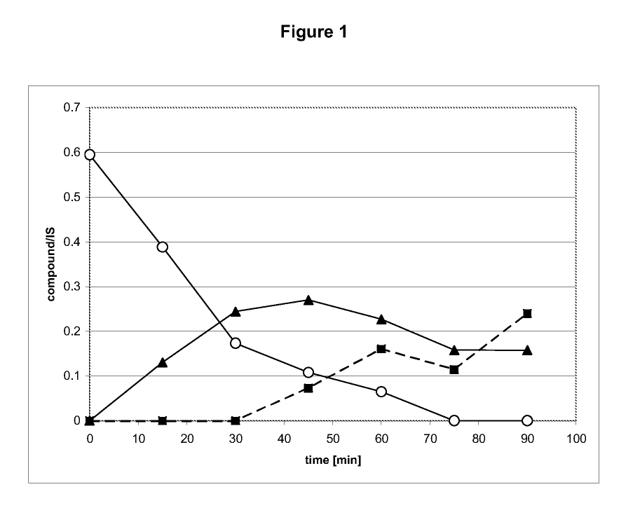

Compound 1 was incubated in human plasma as described in the General Methods—Cleavage Assay above. An aliquot was sampled every 15 min and acidified by the addition of 0.01 mL phosphoric acid to stop the chemical triggered cleavage of the parent compound. The samples were subsequently extracted as described above and analysed immediately using Analysis Method B. The decay of the parent compound and the relative amounts of released compounds are shown in FIG. 1, where the open circles show the decay of 1 and the filled squares and filled triangles indicate the accumulation of macbecin and 18,21-dihydromacbecin respectively.

### Example 8

### Comparison of Cleavage Rates for Compounds 1-5 in Phosphate Buffer and in Whole Blood

Each compound was assessed for cleavage to 18,21-dihydromacbecin as follows. Compounds 1-5 were incubated in whole defribrinated rabbit blood as described in the General Methods—Cleavage Assay above. An aliquot was sampled at 2 min, 1 h, 2.25 h and 3.5 h. 1.5 mL of potassium dihydrogen phosphate was added and the internal standard (IS) to 0.002 mg/mL. The samples were applied to a cartridge described above and analysed immediately using Analysis Method C. T½ values were calculated and are shown in Table 5.

The cleavage of compounds 1-3 was also measured in phosphate buffer as described in the General Methods—Cleavage Assay above at pH 7.2 and pH 7.4 and T_{1/2} values calculated as indicated in Table 5 below.

TABLE 5
Cleavage rates of compounds 1-5

| | 1 | | 2* | | 3 | | 4 | | 5 | |
|---|---|---|---|---|---|---|---|---|---|---|
| | t_{1/2} | R² | t_{1/2} | R² | t_{1/2} | R² | t_{1/2} | R² | t_{1/2} | R² |
| Phosphate Buffer | 64 | >0.99 | 627 | 0.99 | 276 | >0.99 | NT | na | NT | na |
| pH 7.2 (37° C.) | | | | | | | | | | |
| Rabbit blood | 45 | 0.99 | 153 | 0.93 | 160 | 0.81 | NT | na | NT | na |
| pH 7.3 (37° C.) | | | | | | | | | | |
| Run 1 | | | | | | | | | | |
| Rabbit blood | 49 | 0.97 | NT | na | NT | na | 627 | 0.21 | Did | na |
| pH 7.3 (37° C.) | | | | | | | | | not | |
| Run 2 | | | | | | | | | cleave | |
| Phosphate Buffer | 49 | 0.99 | 127 | 0.97 | 160 | 0.97 | NT | na | NT | na |
| pH 7.4 (37° C.) | | | | | | | | | | |

na, not applicable
NT, not tested in the run
*the batch of compound 2 contained impurities which limited accuracy of the analysis

### Example 9

### Solubility Measurements of Compounds 1-3

The compounds 1-3 were tested for their thermodynamic solubility using the method described in the General methods. The results are shown in Table 6 below.

TABLE 6
Solubility of compounds 1-3

| | Mean absorbance | Mean absorbance | Conclusion |
|---|---|---|---|
| | in 5% glucose | in 5% glucose | regarding |
| Compound | (mAU · S) | (mAU · S) | solubility |
| 1 (to 20 mM) | 5122 | 5151 | >20 mM |
| 2 (to 5 mM) | 889 | 907 | >5 mM |
| 2 (to 10 mM) | 1246 | 2432 | <10 mM |
| 3 (to 20 mM) | 4672 | 4291 | >20 mM |

mAU · S, units of quantitative absorbance measurement

### Example 10

### Biological Data—In Vitro Evaluation of Anticancer Activity of Macbecin

In vitro evaluation of macbecin for anticancer activity in a panel of 12 human tumor cell lines in a monolayer proliferation assay was carried out as described in the general methods using a modified propidium iodide assay.

The results are displayed in Table 7 below; each result represents the mean of duplicate experiments.

TABLE 7
In vitro 12 cell line data

Test/Control (%) at drug concentration

| Cell line | 1 × 10−6 M | 1 × 10−5 M |
|---|---|---|
| SF268 | 95.5 | 20.5 |
| 251L | 61 | 37 |
| H460 | 110 | 21.5 |
| MCF7 | 35.5 | 12 |
| MDA231 | 88 | 34 |
| MDA468 | 14.5 | 4 |
| 394NL | 21 | 17 |
| OVCAR3 | 71 | 37.5 |
| DU145 | 42 | 14.5 |
| LNCAP | 43.5 | 27.5 |
| A498 | 107.5 | 82 |
| 1138L | 75.5 | 19 |

### Example 11

### Biological Data—In Vivo Evaluation of 18-O—(N,N′-dimethylethylenediamine-N′-carbamoyl)-18,21-dihydromacbecin Hydrochloride Salt (Compound 1)

In vivo evaluation of 18-O—(N,N′-dimethylethylenediamine-N′-carbamoyl)-18,21-dihydromacbecin hydrochloride 1 was carried out in nude mice bearing xenografts of the human prostate carcinoma cell line DU-145, as described in the general methods. 18-O—(N,N′-dimethylethylenediamine-N′-carbamoyl)-18,21-dihydromacbecin hydrochloride 1 was administered intraperitoneally at a dose level of 60 mg/kg/d on days 0-4, 7-11, 14-18, 21, 24, 25, 28 and 29, in a vehicle of 5% glucose, and compared to a control group dosed with the standard vehicle 10% DMSO, 0.05% Tween 80.

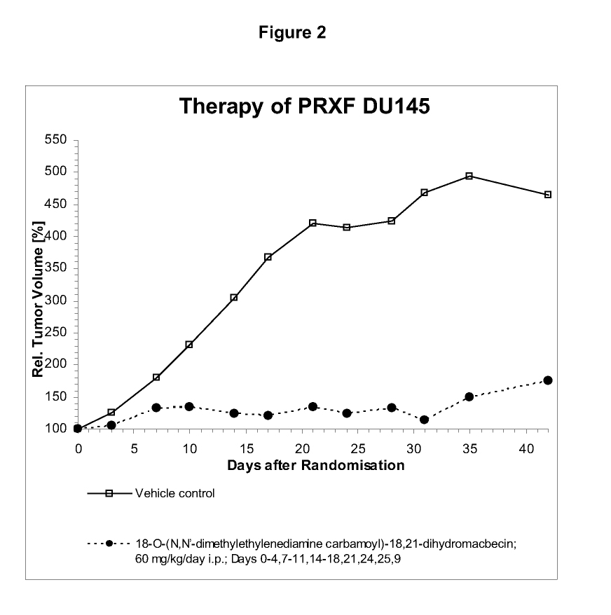

18-O—(N,N′-dimethylethylenediamine-N′-carbamoyl)-18,21-dihydromacbecin hydrochloride 1 at 60 mg/kg/d i.p. on Days 0-4, 7-11, 14-18, 21, 24, 25, 28 and 29 significantly (p<0.001; U-test by Mann-Whitney-Wilcoxon) reduced tumour growth rates (minimum T/C value: 24.3%, recorded on day 31). A graph showing the group median RTV value throughout the course of the study is displayed as FIG. 2.

Antitumour activity was determined as tumour volume inhibition relative to tumours in a control group receiving the standard vehicle 10% DMSO, 0.05% Tween80 in PBS i.p. at 10 mL/kg/d on Days 0-4, 7-11, 14-18, 21, 24, 25, 28 and 29. Group size was 6 mice for the vehicle control group and 6 mice in the therapy groups implanted with two tumour fragments. The experiment was terminated on Day 42.

All references including patent and patent applications referred to in this application are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

### REFERENCES

Allen, I. W. and Ritchie, D. A. (1994) Cloning and analysis of DNA sequences from Streptomyces hygroscopicus encoding geldanamycin biosynthesis. Mol. Gen. Genet. 243: 593-599.

Bagatell, R. and Whitesell, L. (2004) Altered Hsp90 function in cancer: A unique therapeutic opportunity. Molecular Cancer Therapeutics 3: 1021-1030.

Beliakoff, J. and Whitesell, L. (2004) Hsp90: an emerging target for breast cancer therapy. Anti-Cancer Drugs 15:651-662.

Blagosklonny, M. V. (2002) Hsp-90-associated oncoproteins: multiple targets of geldanamycin and its analogues. Leukemia 16:455-462.

Blagosklonny, M. V., Toretsky, J., Bohen, S. and Neckers, L. (1996) Mutant conformation of p53 translated in vitro or in vivo requires functional HSP90. Proc. Natl. Acad. Sci. USA 93:8379-8383.

Bohen, S. P. (1998) Genetic and biochemical analysis of p23 and ansamycin antibiotics in the function of Hsp90-dependent signaling proteins. Mol Cell Biol 18:3330-3339.

Carreras, C. W., Schirmer, A., Zhong, Z. and Santi D. V. (2003) Filter binding assay for the geldanamycin-heat shock protein 90 interaction. Analytical Biochemistry 317:40-46.

Chiosis, G., Huezo, H., Rosen, N., Mimnaugh, E., Whitesell, J. and Neckers, L. (2003) 17AAG: Low target binding affinity and potent cell activity—finding an explanation. Molecular Cancer Therapeutics 2:123-129.

Chiosis, G., Vilenchik, M., Kim, J. and Solit, D. (2004) Hsp90: the vulnerable chaperone. Drug Discovery Today 9:881-888.

Csermely, P. and Soti, C. (2003) Inhibition of Hsp90 as a special way to inhibit protein kinases. Cell. Mol. Biol. Lett. 8:514-515.

DeBoer, C and Dietz, A. (1976) The description and antibiotic production of Streptomyces hygroscopicus var. geldanus. J. Antibiot. 29:1182-1188.

DeBoer, C., Meulman, P. A., Wnuk, R. J., and Peterson, D. H. (1970) Geldanamycin, a new antibiotic. J. Antibiot. 23:442-447.

Dengler W. A., Schulte J., Berger D. P., Mertelsmann R. and Fiebig H H. (1995) Development of a propidium iodide fluorescence assay for proliferation and cytotoxicity assay. Anti-Cancer Drugs, 6:522-532.

Donzé O. and Picard, D. (1999) Hsp90 binds and regulates the ligand-inducible α subunit of eukaryotic translation initiation factor kinase Gcn2. Mol Cell Biol 19:8422-8432.

Egorin M J, Lagattuta T F, Hamburger D R, Covey J M, White K D, Musser S M, Eiseman J L. (2002) “Pharmacokinetics, tissue distribution, and metabolism of 17-(dimethylaminoethylamino)-17-demethoxygeldanamycin (NSC 707545) in CD2F1 mice and Fischer 344 rats.” Cancer Chemother Pharmacol, 49(1), pp 7-19.

Eustace, B. K., Sakurai, T., Stewart, J. K., et al. (2004) Functional proteomic screens reveal an essential extracellular role for hsp90α in cancer cell invasiveness. Nature Cell Biology 6:507-514.

Fang, Y., Fliss, A. E., Rao, J. and Caplan A. J. (1998) SBA1 encodes a yeast Hsp90 cochaperone that is homologous to vertebrate p23 proteins. Mol Cell Biol 18:3727-3734.

Fiebig H. H., Dengler W. A. and Roth T. Human tumor xenografts: Predictivity, characterization, and discovery of new anticancer agents. In: Fiebig H H, Burger A M (eds). Relevance of Tumor Models for Anticancer Drug Development. Contrib. Oncol. 1999, 54: 29-50.

Furniss B. S., Hannaford A. J., Smith P. W. G. and Tatchell A. R. (1989) Vogel's textbook of practical organic chemistry, 5^{th} Ed, Pearson, Prentice Hall, Harlow, UK

Goetz, M. P., Toft, D. O., Ames, M. M. and Ehrlich, C. (2003) The Hsp90 chaperone complex as a novel target for cancer therapy. Annals of Oncology 14:1169-1176.

Grass, G. M., Rubas, W., Jezyk, N., (1992) Evaluation of CACO-2 monolayers as a predictor of drug permeability in colonic tissues. FASEB Journal, 6, A1002.

Harris, S. F., Shiau A. K. and Agard D. A. (2004) The crystal structure of the carboxy-terminal dimerization domain of htpG, the Escherichia coli Hsp90, reveals a potential substrate binging site. Structure 12: 1087-1097.

Hong, Y.-S., Lee, D., Kim, W., Jeong, J.-K. et al. (2004) Inactivation of the carbamoyltransferase gene refines post-polyketide synthase modification steps in the biosynthesis of the antitumor agent geldanamycin. J. Am. Chem. Soc. 126:11142-11143.

Hostein, I., Robertson, D., DiStefano, F., Workman, P. and Clarke, P. A. (2001) Inhibition of signal transduction by the Hsp90 inhibitor 17-allylamino-17-demethoxygeldanamycin results in cytostasis and apoptosis. Cancer Research 61:4003-4009.

Hu, Z., Liu, Y., Tian, Z.-Q., Ma, W., Starks, C. M. et al. (2004) Isolation and characterization of novel geldanamycin analogues. J. Antibiot. 57:421-428.

Hur, E., Kim, H.-H., Choi, S. M., et al. (2002) Reduction of hypoxia-induced transcription through the repression of hypoxia-inducible factor-1α/aryl hydrocarbon receptor nuclear translocator DNA binding by the 90-kDa heat-shock protein inhibitor radicicol. Molecular Pharmacology 62:975-982.

Iwai Y, Nakagawa, A., Sadakane, N., Omura, S., Oiwa, H., Matsumoto, S., Takahashi, M., Ikai, T., Ochiai, Y. (1980) Herbimycin B, a new benzoquinoid ansamycin with anti-TMV and herbicidal activities. The Journal of Antibiotics, 33(10), pp 1114-1119.

Jez, J. M., Chen, J. C.-H., Rastelli, G., Stroud, R. M. and Santi, D. V. (2003) Crystal structure and molecular modeling of 17-DMAG in complex with human Hsp90. Chemistry and Biology 10:361-368.

Kaur, G., Belotti, D, Burger, A. M., Fisher-Nielson, K., Borsotti, P. et al. (2004) Antiangiogenic properties of 17-(Dimethylaminoethylamino)-17-Demethoxygeldanamycin: an orally bioavailable heat shock protein 90 modulator. Clinical Cancer Research 10:4813-4821.

Kumar, R., Musiyenko, A. and Barik S. (2003) The heat shock protein 90 of Plasmodium falciparum and antimalarial activity of its inhibitor, geldanamycin. J Malar 2:30.

Kurebayashi, J., Otsuke, T., Kurosumi, M., Soga, S., Akinaga, S. and Sonoo, H. (2001) A radicicol derivative, KF58333, inhibits expression of hypoxia-inducible factor-1α and vascular endothelial growth factor, angiogenesis and growth of human breast cancer xenografts. Jpn. J. Cancer Res. 92:1342-1351.

Le Brazidec, J.-Y., Kamal, A., Busch, D., Thao, L., Zhang, L. et al. (2003) Synthesis and biological evaluation of a new class of geldanamycin derivatives as potent inhibitors of Hsp90. J. Med. Chem. 47: 3865-3873.

Lee, Y.-S., Marcu, M. G. and Neckers, L. (2004) Quantum chemical calculations and mutational analysis suggest heat shock protein 90 catalyzes trans-cis isomeration of geldanamycin. Chem. Biol. 11:991-998.

Li, A. P. (1992) Screening for human ADME/Tox drug properties in drug discovery. Drug Discovery Today, 6, 357-366

Liu, X.-D., Morano, K. A. and Thiele D. J. (1999); The yeast Hsp110 family member, Sse1, is an Hsp90 cochaperone. J Biol Chem 274:26654-26660.

Mandler, R., Wu, C., Sausville, E. A., Roettinger, A. J., Newman, D. J., Ho, D. K., King, R., Yang, D., Lippman, M. E., Landolfi, N. F., Dadachova, E., Brechbiel, M. W. and Waldman, T. A. (2000) Immunoconjugates of geldanamycin and anti-HER2 monoclonal antibodies: antiproliferative activity on human breast carcinoma cell lines. Journal of the National Cancer Institute 92:1573-1581.

McLaughlin S. H., Smith, H. W. and Jackson S. E. (2002) Stimulation of the weak ATPase activity of human Hsp90 by a client protein. J. Mol. Biol. 315: 787-798.

Muroi M, Izawa M, Kosai Y, Asai M. (1981) “The structures of macbecin I and II” Tetrahedron, 37, pp 1123-1130.

Muroi, M., Izawa M., Kosai, Y., and Asai, M. (1980) Macbecins I and II, New Antitumor antibiotics. II. Isolation and characterization. J Antibiotics 33:205-212.

Neckers, L (2003) Development of small molecule Hsp90 inhibitors: utilizing both forward and reverse chemical genomics for drug identification. Current Medicinal Chemistry 9:733-739.

Neckers, L. (2002) Hsp90 inhibitors as novel cancer chemotherapeutic agents. Trends in Molecular Medicine 8:S55-S61.

Nimmanapalli, R., O'Bryan, E., Kuhn, D., Yamaguchi, H., Wang, H.-G. and Bhalla, K. N. (2003) Regulation of 17-AAG-induced apoptosis: role of Bcl-2, Bcl-x_{L}, and Bax downstream of 17-AAG-mediated down-regulation of Akt, Raf-1, and Src kinases. Neoplasia 102:269-275.

Omura, S., Iwai, Y., Takahashi, Y., Sadakane, N., Nakagawa, A., Oiwa, H., Hasegawa, Y., Ikai, T., (1979), Herbimycin, a new antibiotic produced by a strain of Streptomyces. The Journal of Antibiotics, 32(4), pp 255-261.

Omura, S., Miyano, K., Nakagawa, A., Sano, H., Komiyama, K., Umezawa, I., Shibata, K, Satsumabayashi, S., (1984), “Chemical modification and antitumor activity of Herbimycin A. 8,9-epoxide, 7,9-carbamate, and 17 or 19-amino derivatives”. The Journal of Antibiotics, 37(10), pp 1264-1267.

Ono, Y., Kozai, Y. and Ootsu, K. (1982) Antitumor and cytocidal activities of a newly isolated benzenoid ansamycin, Macbecin I. Gann. 73:938-44.

Rascher, A., Hu, Z., Viswanathan, N., Schirmer, A. et al. (2003) Cloning and characterization of a gene cluster for geldanamycin production in Streptomyces hygroscopicus NRRL 3602. FEMS Microbiology Letters 218:223-230.

Roth T., Burger A. M., Dengler W., Willmann H. and Fiebig H. H. Human tumor cell lines demonstrating the characteristics of patient tumors as useful models for anticancer drug screening. In: Fiebig H H, Burger A M (eds). Relevance of Tumor Models for Anticancer Drug Development. Contrib. Oncol. 1999, 54: 145-156.

Rowlands, M. G., Newbatt, Y. M., Prodromou, C., Pearl, L. H., Workman, P. and Aherne, W. (2004) High-throughput screening assay for inhibitors of heat-shock protein 90 ATPase activity. Analytical Biochemistry 327:176-183

Schulte, T. W., Akinaga, S., Murakata, T., Agatsuma, T. et al. (1999) Interaction of radicicol with members of the heat shock protein 90 family of molecular chaperones. Molecular Endocrinology 13:1435-1488.

Shibata, K., Satsumabayashi, S., Nakagawa, A., Omura, S. (1986a) The structure and cytocidal activity of herbimycin C. The Journal of Antibiotics, 39(11), pp 1630-1633.

Shibata, K., Satsumabayashi, S., Sano, H., Komiyama, K., Nakagawa, A., Omura, S. (1986b) Chemical modification of Herbimycin A: synthesis and in vivo antitumor activities of halogenated and other related derivatives of herbimycin A. The Journal of Antibiotics, 39(3), pp 415-423.

Smith M. B. and March J. (2001) March's advanced organic chemistry, 5^{th} Ed, John Wiley and Sons Inc., UK

Smith-Jones, P. M., Solit, D. B., Akhurst, T., Afroze, F., Rosen, N. and Larson, S. M. (2004) Imaging the pharmacodynamics of HER2 degradation in response to Hsp90 inhibitors. Nature Biotechnology 22:701-706.

Sreedhar A. S., Nardai, G. and Csermely, P. (2004) Enhancement of complement-induced cell lysis: a novel mechanism for the anticancer effects of Hsp90 inhibitors. Immunology letters 92:157-161.

Sreedhar, A. S., Söti, C. and Csermely, P. (2004a) Inhibition of Hsp90: a new strategy for inhibiting protein kinases. Biochimica Biophysica Acta 1697:233-242.

Stead, P., Latif, S., Blackaby, A. P. et al. (2000) Discovery of novel ansamycins possessing potent inhibitory activity in a cell-based oncostatin M signaling assay. J Antibiotics 53:657-663.

Supko, J. G., Hickman, R. L., Grever, M. R. and Malspeis, L (1995) Preclinical pharmacologic evaluation of geldanamycin as an antitumor agent. Cancer Chemother. Pharmacol. 36:305-315.

Takahashi, A., Casais, C., Ichimura K. and Shirasu, K. (2003) HSP90 interacts with RAR1 and SGT1 and is essential for RPS2-mediated disease resistance in Arabidopsis. Proc. Natl. Acad. Sci. USA 20:11777-11782.

Tanida, S., Hasegawa, T. and Higashide E. (1980) Macbecins I and II, New Antitumor antibiotics. I. Producing organism, fermentation and antimicrobial activities. J Antibiotics 33:199-204.

Tian, Z.-Q., Liu, Y., Zhang, D., Wang, Z. et al. (2004) Synthesis and biological activities of novel 17-aminogeldanamycin derivatives. Bioorganic and Medicinal Chemistry 12:5317-5329.

Uehara, Y. (2003) Natural product origins of Hsp90 inhibitors. Current Cancer Drug Targets 3:325-330.

Vasilevskaya, I. A., Rakitina, T. V. and O'Dwyer, P. J. (2003) Geldanamycin and its 17-Allylamino-17-Demethoxy analogue antagonize the action of cisplatin in human colon adenocarcinoma cells: differential caspase activation as a basis of interaction. Cancer Research 63: 3241-3246.

Volpe, D. A., Faustino, P. J., Yu, L. X., (2001) Towards standardisation of an in vitro method of drug absorption. Pharmacopeial Forum, 27, 2916-2922

Watanabe, K., Okuda, T., Yokose, K., Furumai, T. and Maruyama, H. H. (1982) Actinosynnema mirum, a new producer of nocardicin antibiotics. J. Antibiot. 3:321-324.

Wegele, H., Müller, L. and Buchner, J. (2004) Hsp70 and Hsp90-a relay team for protein folding. Rev Physiol Biochem Pharmacol 151:1-44.

Whitesell, L., Mimnaugh, E. G., De Costa, B., Myers, C. E. and Neckers, L. M. (1994) Inhibition of heat shock protein HSP90-pp60^{v-src} heteroprotein complex formation by benzoquinone ansamycins: Essential role for stress proteins in oncogenic transformation. Proc. Natl. Acad. Sci. USA 91: 8324-8328.

Winklhofer, K. F., Heller, U., Reintjes, A. and Tatzelt J. (2003) Inhibition of complex glycosylation increases the formation of PrP^{sc}. Traffic 4:313-322.

Workman P. (2003) Auditing the pharmacological accounts for Hsp90 molecular chaperone inhibitors: unfolding the relationship between pharmacokinetics and pharmacodynamics. Molecular Cancer Therapeutics 2:131-138.

Workman, P. and Kaye, S. B. (2002) Translating basic cancer research into new cancer therapeutics. Trends in Molecular Medicine 8:S1-S9.

Young, J. C.; Moarefi, I. and Hartl, U. (2001) Hsp90: a specialized but essential protein folding tool. J. Cell. Biol. 154:267-273.

1. A derivative of a benzenoid ansamycin which contains a 1,4-dihydroxyphenyl moiety bearing at position 6 an amino carboxy substituent, in which position 2 and the carboxy substituent at position 6 are connected by an aliphatic chain of varying length characterised in that one or both of the 1-hydroxy and the 4-hydroxy position(s) of the phenyl ring are independently derivatised by an aminoalkyleneaminocarbonyl group, which alkylene group, which may optionally be substituted by alkyl groups, has a chain length of 2 or 3 carbons and which derivatising group(s) increase the water solubility and/or the bioavailability of the parent molecule but which are capable of being removed in-vivo.

2. A compound according to Formula (IA-IC) below: 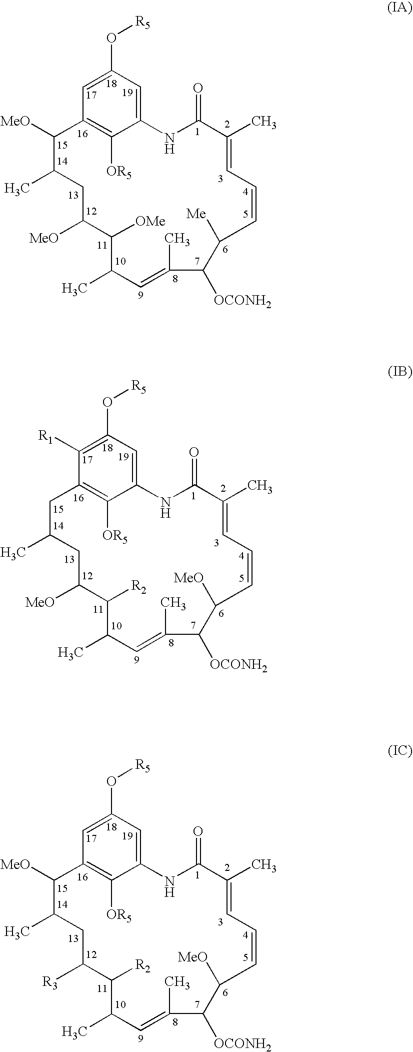
wherein:
R₁ represents H, OH, OMe, —NHCH₂CH═CH₂ or —NHCH₂CH₂N(CH₃)₂;
R₂ represents OH, or keto;
R₃ represents OH or OMe;
R₅ represents H or 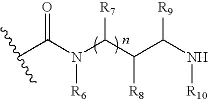
wherein:
n represents 0 or 1;
R₆ represents H, Me, Et or iso-propyl;
R₇, R₈ and R₉ each independently represent H or a C1-C4 branched or linear chain alkyl group; or R₇ and R₈, or R₈ and R₉, may be connected so as to form a 6-membered carbocyclic ring;
R₁₀ represents H or a C1-C4 branched or linear chain alkyl group;
provided however that the R₅ moieties are not both H and that when neither R₅ moiety represents H then the two R₅ moieties are the same; or a pharmaceutically acceptable salt thereof.

3. The compound according to claim 2 wherein R₆ represents H, Me or Et.

4. The compound according to claim 2 wherein R₁₀ represents a C1-C4 branched or linear chain alkyl group.

5. The compound according to claim 2 wherein R₆ represents H, Me or Et and R₁₀ represents a C1-C4 branched or linear chain alkyl group.

6. The compound according to claim 2, wherein neither R₅ represents H.

7. The compound according to claim 2, wherein one R₅ group represents H.

8. The compound according to claim 7 wherein the C21 R₅ group is H.

9. The compound according to claim 2 defined by structure (IA).

10. The compound according to claim 8 defined by structure (IB).

11. The compound according to claim 10 wherein R₁ represents —NHCH₂CH═CH₂.

12. The compound according to claim 10 wherein R₁ represents —NHCH₂CH₂N(CH₃)₂.

13. The compound according to claim 10 wherein R₁ represents OMe.

14. The compound according to claim 2 defined by structure (IC).

15. The compound according to claim 2 wherein n is 0.

16. The compound according to claim 2 wherein R₆ represents Me.

17. The compound according to claim 2 wherein R₆ represents Et.

18. The compound according to claim 2 wherein R₁₀ represents Me.

19. The compound according to claim 2 wherein R₁₀ represents Et.

20. The compound according to claim 2 wherein R₇ represents H.

21. The compound according to claim 2 wherein R₈ and R₉ represent H.

22. The compound according to claim 2 which is 18-O—(N,N′-dimethylethylenediamine-N′-carbamoyl)-18,21-dihydromacbecin or a pharmaceutically acceptable salt thereof.

23. The compound according to claim 2 selected from:
18-O—(N-methylethylenediamine-N′-carbamoyl)-18,21-dihydromacbecin;
18-O—(N,N′-diethylethylenediamine-N′-carbamoyl)-18,21-dihydromacbecin;
18-O—(N,N′-dimethyl-1,3-propanediamine-N′-carbamoyl)-18,21-dihydromacbecin; and
18-O—(N,N′-diisopropylethylenediamine-N′-carbamoyl)-18,21-dihydromacbecin
or a pharmaceutically acceptable salt of any one thereof.

24. The compound according to claim 1 in the form of a hydrochloride salt.

25. (canceled)

26. (canceled)

27. A pharmaceutical composition comprising a compound according to claim 1 together with one or more pharmaceutically acceptable diluents or carriers.

28. A method of treatment of cancer or B-cell malignancies which comprises administering to a patient an effective amount of a compound according to claim 1.

29. (canceled)

30. A process for preparing a compound of formula (I) or a pharmaceutically acceptable salt thereof which comprises:
(a) preparing a compound of formula (I) in which neither R₅ moiety is H by reacting a compound of formula (IIA), (IIB) or (IIC): 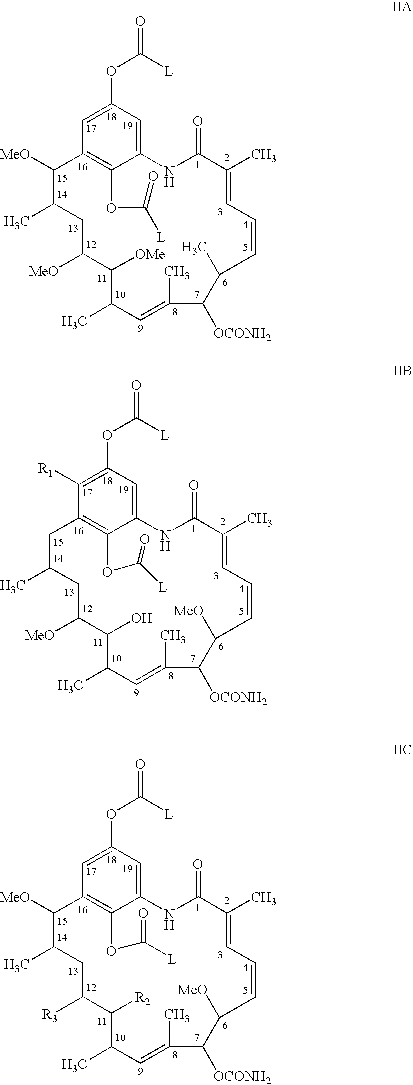
wherein R₁, R₂ and R₃ are as defined in claim 2 and L is a leaving group, or a protected derivative thereof, with a compound of formula (H) 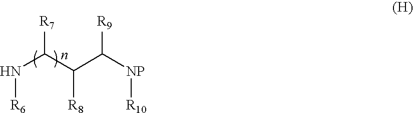
wherein n, R₆, R₇, R₈, R₉ and R₁₀ are as defined in claim 2 and P represents a protecting group; or
(b) preparing a compound of formula (I) in which the C21 R₅ moiety is H by reacting a compound of formula (IID), (IIE) or (IIF): 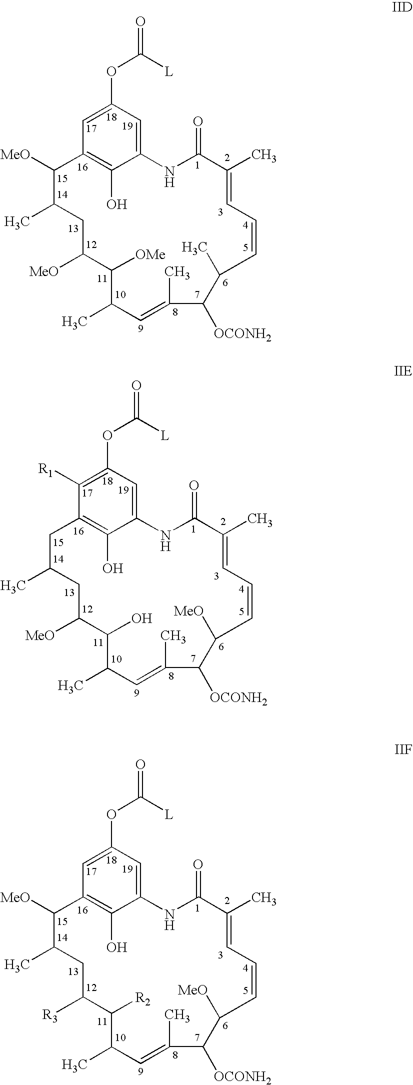
wherein R₁, R₂ and R₃ are as defined in claim 2 and L is a leaving group
or a protected derivative thereof, with a compound of formula (H) 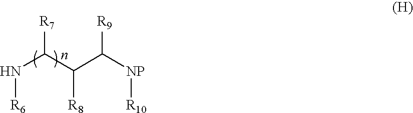
wherein n, R₆, R₇, R₈, R₉ and R₁₀ are as defined in claim 2 and P represents a protecting group; or
(c) converting a compound of formula (I) or a salt thereof to another compound of formula (I) or another pharmaceutically acceptable salt thereof; or
(d) deprotecting a protected compound of formula (I).

31. A compound of formula (IIA), (IIB) or (IIC): 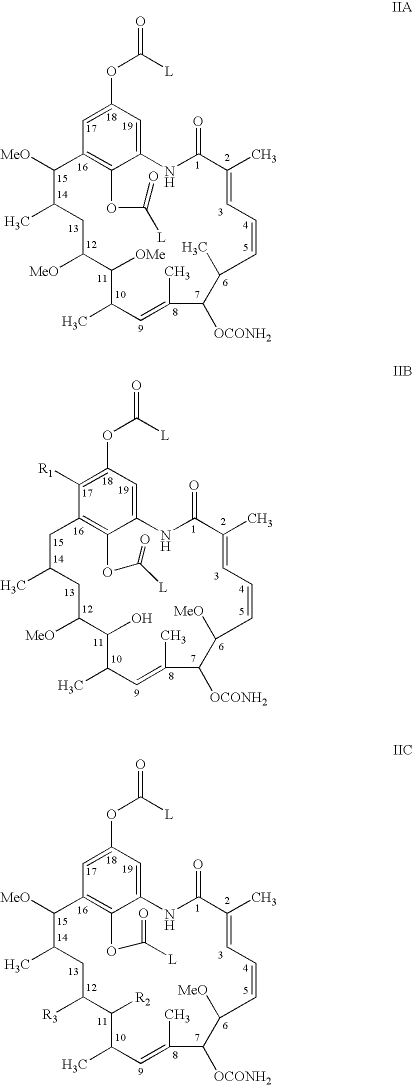
wherein R₁, R₂ and R₃ are as defined in claim 2 and L is a leaving group, or a protected derivative of any one thereof.

32. A compound of formula (IID), (IIE) or (IIF) 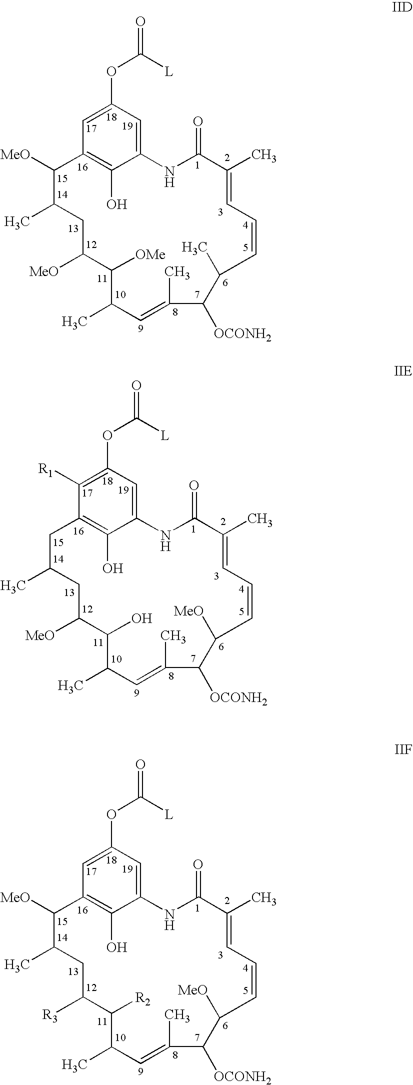
wherein R₁, R₂ and R₃ are as defined in claim 2 and L is a leaving group
or a protected derivative of any one thereof.

33. A compound of formula (IVA), (IVB) or (IVC) 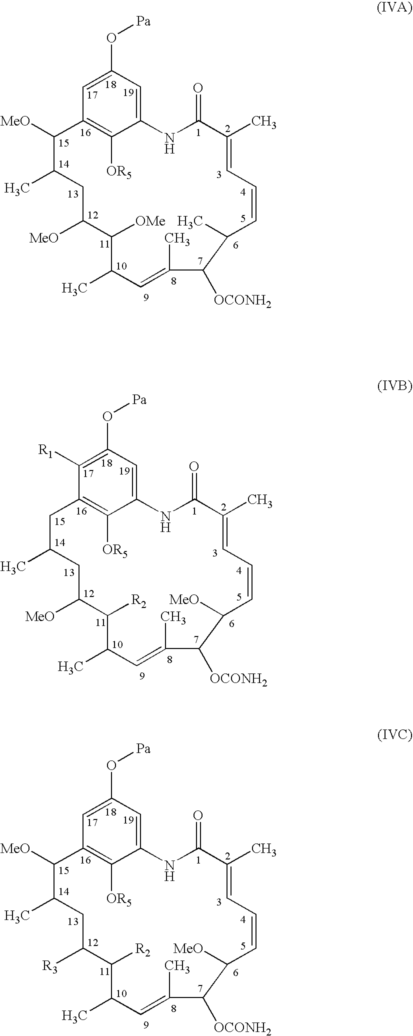
wherein R₁, R₂, R₃ and R₅ are as defined in claim 2 and Pa is a protecting group, or a protected derivative of any one thereof.

34. A compound of formula (VA), (VB) or (VC) 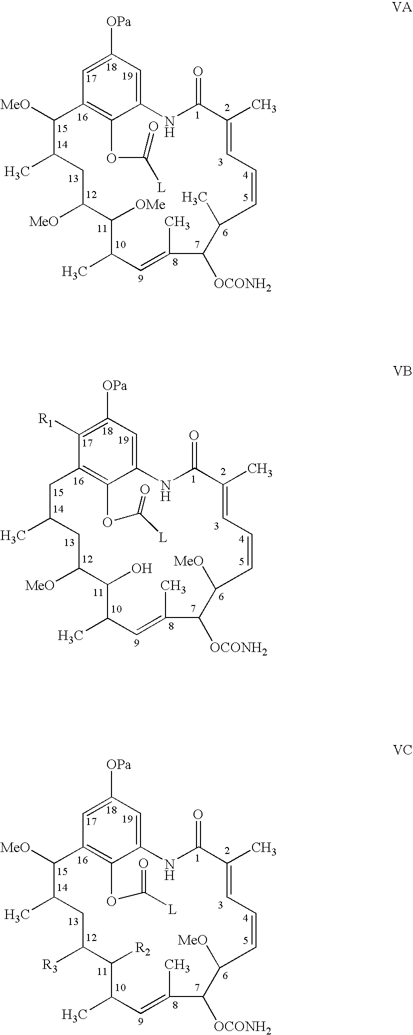
wherein R₁, R₂ and R₃ are as defined in claim 2, L is a leaving group and Pa is a protecting group, or a protected derivative of any one thereof.

35. The compound according to claim 2 in the form of a hydrochloride salt.

36. A pharmaceutical composition comprising a compound according to claim 2 together with one or more pharmaceutically acceptable diluents or carriers.

37. A method of treatment of cancer or B-cell malignancies which comprises administering to a patient an effective amount of a compound according to claim 2.
